(19) United States
(12) Patent Application Publication
(10) Pub. No.: US 2026/0084978 A1
(43) Pub. Date: Mar. 26, 2026

(54) PROCESS FOR PRODUCING ATOMIC QUANTUM CLUSTERS DERIVATIVES

(71) Applicants: NANOGAP SUB-NM-POWDER, S.A., Ames, A Coruna (ES); UNIVERSIDADE DE SANTIAGO DE COMPOSTELA, Santiago de Compostela, A Coruna (ES)

(72) Inventors: David BUCETA FERNÁNDEZ, Ames (ES); Manuel Arturo LÓPEZ QUINTELA, Santiago de Compostela, A Coruna (ES)

(21) Appl. No.: 19/111,206

(22) PCT Filed: Sep. 15, 2023
(86) PCT No.: PCT/EP2023/075494

§ 371 Date: Mar. 12, 2025

(30) Foreign Application Priority Data
Sep. 15, 2022 (EP) 22382850.0

(51) Int. Cl. C01G 5/00; A61K 33/38; A61K 51/00; A61P 35/04; B01J 23/66; B01J 23/72; C01G 3/00

(52) U.S. Cl. CPC C01G 5/00; A61K 33/38; A61K 51/00; A61P 35/04; B01J 23/66; B01J 23/72; C01G 3/00; A61K 2121/00

(57) ABSTRACT

The present invention provides an easy and scalable process for producing a compound of formula (I): N_{(2y/z)}[Mₓ(GO₃)_{y}](I), wherein [Mₓ(GO₃)_{y}] is an anion, wherein Mₓ is an Atomic Quantum Cluster (AQC) consisting of x number of zero valent metal atoms; wherein G is Si or Ti; and y is an integer selected from 1, 2, 3, 4, 5 and 6; and wherein the compound of formula (I) has no net charge. In addition, the present invention is directed to a composition comprising the compound of formula (I), a process for producing the compound of formula (I); the uses of the compound of formula (I) and the compound of formula (I) for use as a medicament, its second medical uses, a pharmaceutical composition, kit-of-parts and an apoptotic agent comprising said compound of formula (I).

### FIELD OF THE ART

The present invention relates to a process for producing atomic quantum clusters (AQCs) derivatives.

### STATE OF THE ART

The high catalytic activity of atomic quantum clusters (AQCs) when compared with isolated atoms or nanoparticles is well known in the state of the art [A. Corma et al., Nature Chemistry, vol. 5, p. 775-781, 2013]. Due to the potential applications of the atomic quantum clusters (AQCs) in the field of biosensors, electrocatalysis, magnetism, photoluminescence or catalysis, the development of synthesis methods for producing AQCs has arisen a great interest. There are several methods for synthesizing stable AQCs and AQCs derivatives: i) top-down approaches by etching small nanoparticles with an excess of strong binding ligands; and ii) bottom-up approaches using strong binding ligands to inhibit the growth of the AQCs usually employing strong reducing agents. The use of organic ligands is usually required in both approaches. However, organic ligands may hinder some of the important properties of the AQCs, such as their catalytic properties. In addition, the AQC can catalyze oxidation of the organic group of the ligand, preventing its attachment. Moreover, an AQC comprising organic ligands easily degrades at temperatures over 100-200° C. and can lead to cluster aggregation.

Therefore, despite the reported synthesis methods, there is still a need in the art for a new simple and scalable method for producing different AQCs derivatives in high concentrations and with a high yield.

### BRIEF DESCRIPTION OF THE INVENTION

The authors of the present invention have synthesized a compound of formula (I) that comprises AQCs and inorganic ligands such as titanates or silicates, which overcomes the drawbacks of AQCs comprising organic ligands. In particular, the compound of formula (I) that comprises AQCs and inorganic ligands is much more resistant to aggregation and agglomeration than bare AQCs or AQCs with organic ligands, thus, solutions with high concentration of AQCs derivatives can be made. In addition, the use of inorganic ligands such as titanate or silicate groups does not hinder the catalytic properties of the AQCs. Moreover, the compounds of formula (I) that comprise AQCs and inorganic ligands, is stable at temperatures up to 700° C. or even higher without losing their physicochemical and biological properties. As a result, the spectrum of catalytic and therapeutic possible applications of said compounds is significantly enhanced/increased.

In addition, authors of the present invention have developed a new method of synthesis of compound of formula (I) that comprises AQCs and inorganic ligands that shows a higher product yield than synthesis methods without the presence of those ligands.

A first aspect of the invention is directed to a compound of formula (I)

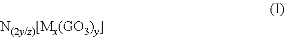

wherein:
   N is at least a cation with one or two positive charges; and
   z is 1 or 2; and
wherein [Mₓ(GO₃)_{y}] is an anion, wherein:
   Mₓ is an Atomic Quantum Cluster (AQC) consisting of x number of zero valent metal atoms; optionally wherein M is selected from Ag, Cu, Co, Au, Pt, Fe, Pd, Ni or their bi-metal and multi-metal combinations; and wherein x is an integer selected from 2, 3, 4, 5, 6, 7, 8, 9 and 10; and
   G is Si, Ti or a combination thereof; and
   y is an integer selected from 1, 2, 3, 4, 5 and 6; and
wherein the compound of formula (I) has no net charge.

In a second aspect, the invention refers to an anion consisting of:
Atomic Quantum Clusters (AQCs) consisting of 2, 3, 4, 5, 6, 7, 8, 9 and 10 zero valent metal atoms; wherein the metal atoms are selected from Ag, Co, Cu, Au, Pt, Fe, Pd, Ni or their bi-metal and multi-metal combinations; and
an “y” number of anionic ligands of formula (GO₃²⁻) wherein G is Si or Ti; preferably selected from a metasilicate (SiO₃²⁻) or a metatitanate (TiO₃²⁻); wherein y is an integer selected from 1, 2, 3, 4, 5 and 6.

Further, in a third aspect the invention refers to a process for producing the compound of formula (I) comprising the following steps:
i. providing:
   a first solution comprising:
      a polar solvent, and
      Atomic Quantum Clusters (AQCs) of formula (II)

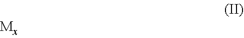

wherein Mₓ is an Atomic Quantum Cluster (AQC) consisting of x number of zero valent metal atoms; optionally wherein M is selected from Ag, Cu, Co, Au, Pt, Fe, Pd, Ni or their bi-metal and multi-metal combinations; and wherein x is an integer selected from 2, 3, 4, 5, 6, 7, 8, 9 and 10; and
a precursor compound; wherein the precursor compound comprises at least one of Si or Ti;
ii. adding the precursor to the solution of step (i) to obtain a second solution;
iii. optionally repeating step (i) and/or step (ii);
wherein the molar ratio between the precursor added in each step and the AQCs of the solution of previous step are in the range of between 0.1 to 10 equivalents.

In a further aspect, the invention is directed to the use of the compound of formula (I) as catalyst.

In an additional aspect, the invention is directed to the compound of formula (I) for use as a medicament.

### FIGURES

The accompanying drawings, which are incorporated herein and constitute a part of this specification, illustrate exemplary embodiments of the invention and, together with the written description, serve to explain the principles of the invention. In the drawings:

FIG. 1: Calculation of the stability of compounds [Cu₅SiO₃]²⁻ and [Cu₅(SiO₃)₂]⁴⁻.

FIG. 2: Sketch of the structure of compounds [Cu₅SiO₃]²⁻ and [Cu₅(SiO₃)₂]⁴⁻

FIG. 3: % viability vs dose response curve for A549 cells results for (i) Ag₅-silicate atomic quantum clusters compounds such as Na₂[Ag₅SiO₃] or Na₄ [Ag₅(SiO₃)₂], (ii) Ag+ as control and (iii) Cisplatin.

FIG. 4 shows the % viability response data for A549 cells results for Ag+ used as control, different concentrations of selumetinib, sotorasib, and Ag₅-silicate atomic quantum clusters compounds at different concentrations.

FIG. 5 shows % of inhibition test results on NCI-H358 cells.

FIG. 6 shows % of inhibition test results on NCI-H23 cells.

FIG. 7 shows the surviving fraction results vs radiation dose of combining five atoms Ag₅-silicate atomic quantum clusters compounds with an external beam radiation.

FIG. 8 shows the survival % vs days since the cell line injection results for the control sample (with no treatment), for the historical control and for the sample treated with Ag5-silicate atomic quantum clusters compounds.

FIG. 9 shows the RLU (μg protein) results for the A549-luc cells of the control (no treatment) samples and of the samples treated with Cisplatin (4 mg/kg) and with Ag5-silicate atomic quantum clusters compounds (0.25 mg/kg).

FIG. 10 shows: (a) the % of inhibition results for NCI-H358 cells and (b) for NCI-H23 cells non treated, treated with Ag+ as control, treated with BI-3406 (Sos1 inhibitor) in an amount of 10 μM for 24 hours, five atoms Ag₅-silicate atomic quantum clusters at different concentrations (2.6 μM and 4 μM) for 1 hour, a combination of BI-3406 and five atoms Ag₅-silicate atomic quantum clusters at different concentrations and a combination of a combination of BI-3406 and sotorasib.

FIG. 11 shows the viability % vs the Ag5-silicate atomic quantum clusters compounds concentration in micromoles (μM) for a A549 cell line in comparison with the viability results % of 2 μM of selumetinib, AZ and of the combination of 2 μM of selumetinib and Ag5-silicate atomic quantum clusters compounds.

FIG. 12 shows the viability % vs the Ag5-silicate atomic quantum clusters compounds concentration in micromoles (μM) for a H359 cell line in comparison with the viability results % of 100 nM of sotorasib and of the combination of 100 nM of sotorasib and Ag5-silicate atomic quantum clusters compounds.

FIG. 13 shows the results for tumor size (%) over in vivo monitoring tumor grow for a control sample and a sample treated with Ag5-silicate atomic quantum clusters compounds on a U87 Orthotopic in vivo model.

FIG. 14 shows the results of the % of live cells vs the log 10 of μM of Ag5-silicate atomic quantum clusters compounds in an in vitro treatment of a patient derived Glioblastoma Multiforme (GBM) cell lines (20+ lines now tested).

FIG. 15 shows the cell viability % over the Ag5-silicate atomic quantum clusters compounds micro molar concentration (μM) in patient derived esophageal cancer cell line (KYSE350)

FIG. 16 shows the cell viability (%) over the Ag5-silicate atomic quantum clusters compounds micro molar concentration of a 72 h treatment by Dunnett's test.

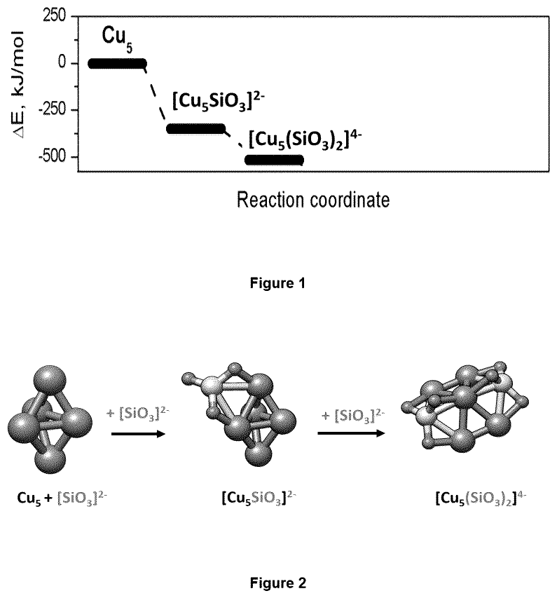

### DETAILED DESCRIPTION OF THE INVENTION

With regard to the terms used in the present description, unless otherwise defined, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

As used herein, the terms “about” or “around” means a slight variation of the value specified, preferably within 10 percent of the value specified. Nevertheless, the term “about” or the term “around” can mean a higher tolerance of variation depending on for instance the experimental technique used. Said variations of a specified value are understood by the skilled person and are within the context of the present invention. Further, to provide a more concise description, some of the quantitative expressions given herein are not qualified with the term “about”. It is understood that, whether the term “about” is used explicitly or not, every quantity given herein is meant to refer to the actual given value and it is also meant to refer to the approximation to such given value that would reasonably be inferred based on the ordinary skill in the art, including equivalents and approximation due to the experimental and/or measurement conditions for such given value.

Throughout the specification, unless the context requires otherwise, the term “consisting essentially of”, and variations such as “consists essentially of”, will be understood to imply the inclusion of a stated integer, step, group of integers or group of steps, with the exclusion of any other integer, step, group of integers or group of steps which materially affects the essential characteristics of the stated integer, step, group of integers or group of steps.

The term “substantially free of” may be used to refer to a composition which is mostly or completely free of an entity specifically mentioned thereafter, or at least does not contain the entity in an amount such that the entity affects the efficacy, storability, usability regarding necessary safety concerns, and/or stability of the composition.

A first aspect of the invention is directed to a compound of formula (I)

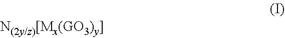

wherein:
   N is at least a cation with one or two positive charges; and
   z is 1 or 2; and
wherein [Mₓ(GO₃)_{y}] is an anion wherein
   Mₓ is an Atomic Quantum Cluster (AQC) consisting of x number of zero valent metal atoms; optionally wherein M is selected from Ag, Cu, Co, Au, Pt, Fe, Pd, Ni or their bi-metal and multi-metal combinations; and wherein x is an integer selected from 2, 3, 4, 5, 6, 7, 8, 9 and 10; and
   G is Si, Ti or a combination thereof; and
   y is an integer selected from 1, 2, 3, 4, 5 and 6; and
      wherein the compound of formula (I) has no net charge.

In an embodiment, N of the compound of formula (I), is a cation with one or two positive charges; preferably is a metal cation; more preferably is an alkali metal cation, an alkaline earth metal cation or combinations thereof; even more preferably is selected from the group consisting of Li⁺, Na⁺, K⁺, Ca²⁺, Mg²⁺ and combinations thereof; preferably is Na⁺ or K⁺.

In an embodiment, z of the compound of formula (I) is an integer selected from one or two.

In an embodiment, [Mₓ(GO₃)_{y}] of the compound of formula (I) is an anion, i.e. comprises a negative charge or is negatively charged.

In a more particular embodiment, [Mₓ(GO₃)_{y}] of the compound of formula (I) consists of an Atomic Quantum Cluster (AQC) consisting of 2, 3, 4, 5, 6, 7, 8, 9 or 10 zero valent metal atoms comprising (GO₃) anionic ligands; wherein G is Si, Ti or a combination thereof; and y is an integer selected from 1, 2, 3, 4, 5 and 6; preferably wherein (GO₃) are SiO₃²⁻ or TiO₃²⁻.

In an embodiment, the [Mₓ(GO₃)_{y}] of the compound of formula (I) is an anion that comprises:
an Atomic Quantum Cluster (AQC) consisting of 2, 3, 4, 5, 6, 7, 8, 9 or 10 zero valent metal atoms selected from Ag, Co, Cu, Au, Pt, Fe, Pd, Ni or their bi-metal and multi-metal combinations; and
at least an inorganic ligand, such as SiO₃²⁻ or TiO₃²⁻; preferably between 1 and 6 inorganic ligands.

In a particular embodiment, the compound of formula (I) has no net charge (it is not charged); preferably the net or total charge of said compound is 0.

In an embodiment, Mₓ of the [Mₓ(GO₃)_{y}] anion of the compound of formula (I) is an Atomic Quantum Cluster (AQC) consisting of x number of zero valent metal atoms. In the context of the present invention, the term “cluster” refers to nanometric and/or sub-nanometric species consisting of well-defined structures of metal atoms with sizes below approximately 1-2 nm. Due to quantum effects, the clusters present discrete energy levels and an increasing band gap as the size of the AQCs decreases.

In the context of the present invention, the term “atomic quantum cluster” or “AQC” means, in accordance with the present invention, a group of two or more zero-valent metal atoms; preferably of zero-valent transition metal atoms. The atomic quantum clusters are also known as “metal quantum clusters” in the state of the art. In an embodiment, the AQCs consist of identical (mononuclear clusters) or different (heteronuclear clusters) zero-valent transition metals. The term “metal” in the context of the present invention refers to the elements of the periodic table known as “metal”, particularly “transition metal”, but it does not refer to the electrical behavior of said elements. The confinement of electrodes in the AQCs originates the quantum separation of the energy levels producing important changes in the properties of these materials. Thus, the metal atoms in the AQCs have a semiconductor-like or even insulating-like behavior.

The AQCs of the compound of formula (I) of the present invention are represented as “Mₓ”, wherein “M” represents a zero-valent metal element, and “x” represents the number of atoms of the zero-valent metal element of the AQCs.

In an embodiment, the number of atoms x of Mₓ is less than 100 atoms, preferably of less than 50; more preferably less than 40; even more preferably less than 30; even much more preferably less than 20 or less than 10.

In an embodiment, the number of atoms x of Mₓ is equal or more than 2 and equal or less than 40; preferably equal or more than 2 and equal or less than 30; more preferably is equal or more than 2 and equal or less than 15.

In a more particular embodiment, the number of atoms x of Mₓ is an integer selected from 2, 3, 4, 5, 6, 7, 8, 9 and 10; more preferably selected from 3, 4, 5, 6, 7, 8, 9 and 10; preferably selected from 3, 4, 5, 6, 7 and 8; more preferably of 4, 5, 6 and 7; even much more preferably of 4, 5 and 6.

In an embodiment, the average size of the Atomic Quantum Cluster (AQC), Mₓ, of the invention is of less than 2 nm; preferably less than 1.5 nm; more preferably less than 1 nm. In the context of the present invention, the average size of an AQC might be calculated from a significant number of measurements of methods known in the art such as microscopic, spectroscopic and mass spectrometry methods.

In an embodiment, the zero-valent metal element M of the AQCs with formula Mₓ, is selected from Ag, Cu, Co, Au, Pt, Fe, Pd, Ni or their bi-metal and multi-metal combinations; preferably wherein M is selected from Ag, Cu, Au and Pt or their bi-metal and multi-metal combinations; more preferably wherein M is Ag, Cu or their bi-metal combinations; even more preferably is Ag or Cu.

In an embodiment, (GO₃)_{y} of the compound of formula (I) is an inorganic ligand; in particular, (GO₃)_{y} is joined to Mₓ; preferably is covalently joined to Mₓ.

In an embodiment, (GO₃)_{y} of the compound of formula (I) is an ionic compound; preferably selected from silicate or titanate compounds; more preferably from an y number of metasilicate (SiO₃²⁻) or metatitanate (TiO₃²⁻) compounds; more preferably wherein y is an integer selected from 1, 2, 3, 4, 5 and 6 that preferably indicates the number of metasilicate or metatitanate ions in the compound of formula (I).

In a particular embodiment, the group (GO₃)_{y} of the compound of formula (I) is a ligand of the AQCs, Mₓ; preferably is an inorganic ligand; more preferably is an anionic inorganic ligand.

In a particular embodiment, the invention is directed to a compound of formula (I)

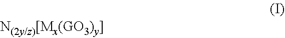

wherein:
   N is selected from Li⁺, Na⁺, K⁺, Ca²⁺, Mg²⁺ or combinations thereof; and
   z is 1 or 2; and
wherein [Mₓ(GO₃)_{y}] is an anion wherein
   Mₓ is an Atomic Quantum Cluster (AQC) consisting of x number of zero valent metal atoms; wherein M is selected from Ag, Cu, Co, Au, Pt, Fe, Pd, Ni or their bi-metal and multi-metal combinations; and wherein x is an integer selected from 2, 3, 4, 5, 6, 7, 8, 9 and 10; and
   (GO₃) is an anionic compound selected from (SiO₃²⁻) or (TiO₃²⁻); and y is an integer selected from 1, 2, 3, 4, 5 and 6; and
      wherein the compound of formula (I) has no net charge.

In an embodiment, the compound of formula (I) comprises:
an Atomic Quantum Cluster (AQC) consisting of 2, 3, 4, 5, 6, 7, 8, 9 or 10 zero valent metal atoms selected from Ag, Co, Cu, Au, Pt, Fe, Pd, Ni or their bi-metal and multi-metal combinations;
at least an anionic inorganic ligand; preferably between 1 and 6 anionic inorganic ligands; more preferably wherein the anionic inorganic ligand is a titanate, silicate or a mixture thereof such as SiO₃²⁻, TiO₃²⁻ or mixtures thereof; and
at least a counterion, preferably a cation; more preferably a cation selected from Li⁺, Na⁺, K⁺, Ca²⁺, Mg²⁺ or combinations thereof;
   wherein the compound of formula (I) has no net charge.

In the context of the present invention the compound of formula (I) may be referred to as an Atomic Quantum Cluster (AQC) derivative.

In an embodiment, the compound of formula (I) consists of:
an Atomic Quantum Cluster (AQC) consisting of 2, 3, 4, 5, 6, 7, 8, 9 or 10 zero valent metal atoms selected from Ag, Co, Cu, Au, Pt, Fe, Pd, Ni or their bi-metal and multi-metal combinations;
between 1 and 6 SiO₃²⁻ and/or TiO₃²⁻ ligands; and
at least a counterion, preferably selected from Li⁺, Na⁺, K⁺, Ca²⁺, Mg²⁺ or combinations thereof;
   wherein said compound of formula (I) has no net charge.

### Anion

An aspect of the invention is directed to an anion consisting of:
Atomic Quantum Clusters (AQCs) consisting of 2, 3, 4, 5, 6, 7, 8, 9 and 10 zero valent metal atoms; wherein the metal atoms are selected from Ag, Co, Cu, Au, Pt, Fe, Pd, Ni or their bi-metal and multi-metal combinations; and
an “y” number of anionic ligands of formula (GO₃²⁻) wherein G is Si or Ti; preferably selected from a metasilicate (SiO₃²⁻) or a metatitanate (TiO₃²⁻); wherein y is an integer selected from 1, 2, 3, 4, 5 and 6.

In an embodiment, the anion comprises:
an Atomic Quantum Cluster (AQC) consisting of 2, 3, 4, 5, 6, 7, 8, 9 or 10 zero valent metal atoms selected from Ag, Co, Cu, Au, Pt, Fe, Pd, Ni or their bi-metal and multi-metal combinations; and
at least an inorganic ligand, such as SiO₃²⁻, TiO₃²⁻ or mixtures thereof; preferably between 1 and 6 inorganic ligands.

In an embodiment, the anion consists of:
an Atomic Quantum Cluster (AQC) consisting of 2, 3, 4, 5, 6, 7, 8, 9 or 10 zero valent metal atoms selected from Ag, Co, Cu, Au, Pt, Fe, Pd, Ni or their bi-metal and multi-metal combinations; and
at least an inorganic ligand, such as SiO₃²⁻, TiO₃²⁻ or mixtures thereof; preferably between 1 and 6 inorganic ligands.

### Process

An aspect of the invention is directed to a process for producing a compound of formula (I) comprising the following steps:
i. providing:
   a first solution comprising:
      a polar solvent, and
      Atomic Quantum Clusters (AQCs) of formula (II)

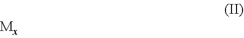

wherein Mₓ is an Atomic Quantum Cluster (AQC) consisting of “x” number of zero valent metal atoms “M”; optionally, wherein M is selected from Ag, Cu, Co, Au, Pt, Fe, Pd, Ni or their bi-metal and multi-metal combinations; and wherein x is an integer selected from 2, 3, 4, 5, 6, 7, 8, 9 and 10; and
a precursor compound; wherein the precursor compound comprises at least one of Si or Ti;
ii. adding the precursor to the solution of step (i) to obtain a second solution;
iii. optionally repeating step (i) and/or step (ii); and
   wherein the molar ratio between the precursor added in each step and the AQCs of the solution of previous step are in the range of between 0.1 to 10 equivalents.

In a particular embodiment, the polar solvent of step (i) is selected from water, methanol, ethanol, acetonitrile, chloroform, dichloromethane, acetic acid, toluene and mixtures thereof; preferably is water; more preferably is milli Q water.

Suitable Atomic Quantum Clusters (AQCs) of formula (II) of step (i) include any AQC available in the market or obtained in the laboratory by methods known in the art.

Moreover, some metal salts available in the market can already contain small amounts of AQCs, which can act as starting AQCs (Peyser, L. A.; Vinson, A. E.; Bartko, A. P.; Dickson, R. M. Science 2001, 291, 103-106). However, a strict control of the amount of clusters present in the metal salt is recommended in order to get reproducible results.

In a particular embodiment, the Atomic Quantum Clusters (AQCs) of step (i) comprise “x” number of zero valent metal atoms “M”; wherein M is selected from Ag, Cu, Co, Au, Pt, Fe, Pd, Ni or their bi-metal and multi-metal combinations; and wherein x is an integer selected from 2, 3, 4, 5, 6, 7, 8, 9 and 10.

In a particular embodiment, the Atomic Quantum Clusters (AQCs) of formula (II) of step (i) consist of “x” number of zero valent metal atoms “M”; wherein M is selected from Ag, Cu, Co, Au, Pt, Fe, Pd, Ni or their bi-metal and multi-metal combinations; and wherein x is an integer selected from 2, 3, 4, 5, 6, 7, 8, 9 and 10.

The Atomic Quantum Clusters (AQCs) of formula (II) of step (i) of the method of the invention have the same advantages and characteristics that those AQCs of formula Mₓ defined above for the compound of formula (I) of the invention, including all their particular embodiments.

In a particular embodiment, the precursor compound of step (i) comprises at least one element selected form Si, Ti or a combination thereof, preferably the precursor compound comprises O and at least one of Si or T; more preferably is a silicate and/or a titanate; even more preferably is a monosilicate and/or monotitanate; much more preferably is a alkaline and/or alkaline earh monosilicate or monotitanate; even much more preferably is a sodium monosilicate and/or monotitanate.

In a particular embodiment, the first solution of step (i) is obtained by a process comprising the following steps:
a. providing
   a metal electrode; optionally wherein the metal is selected from Ag, Cu, Co, Au, Pt, Fe, Pd, Ni or their bi-metal and multi-metal combinations; and
   a solution comprising a polar solvent;
wherein the electrode is in contact with the solution;
b. applying an electric current for at least 50 s to the electrode of step (a) to obtain a first solution comprising a polar solvent and Atomic Quantum Clusters (AQCs) of formula (II).

In a particular embodiment, the metal of the electrode of step (a) is selected from Ag, Cu, Co, Au, Pt, Fe, Pd, Ni or their bi-metal and multi-metal combinations; preferably is selected from Ag, Cu, Au and Pt or their bi-metal and multi-metal combinations; more preferably is Ag, Cu or their bi-metal combinations; even more preferably is Ag or Cu.

In a particular embodiment, the metal of the electrode of step (a) does not comprise oxides.

In a particular embodiment, the metal electrode of step (a) is a polished metal electrode; preferably polished in wet conditions.

In a particular embodiment, the metal electrode of step (a) is part of a cell further comprising a working electrode and a reference electrode; and wherein the electric current of step (b) is obtained by applying an electrical potential difference between the working and the reference electrode. The working electrode and the reference electrode of step (b) can by any electrode known in the art.

In a particular embodiment, the reference electrode is a normal hydrogen electrode (NHE).

In a more particular embodiment, the electrical potential difference between the working and the reference electrode is between 0.1 and 15 V; preferably between 0.2 and 10 V; more preferably between 0.5 and 8 V; even more preferably between 1 and 3 V; even much more preferably between 1.1 and 2.5V; more preferably at about 1.5 V.

In a more particular embodiment, the electrical potential difference between the working and the reference electrode is applied during more than 50 seconds; preferably more than 100 seconds; more preferably more than 200 seconds; even more preferably more than 300 seconds.

In a more particular embodiment, the electrical potential difference between the working and the reference electrode is applied during between 50 and 2000 seconds; preferably between 100 and 1500 seconds; more preferably between 200 and 1000 seconds; even more preferably between 300 and 800 seconds; much more preferably for between 400 and 600 seconds.

In a more particular embodiment, the electric current of step (b) is less than 20 A/cm²; preferably less than 19 A/cm²; more preferably less than 18 A/cm².

In a more particular embodiment, the electric current of step (b) is between 0.01 and 20 A/cm²; preferably between 0.05 and 19 A/cm²; more preferably between 0.08 and 18 A/cm².

In a particular embodiment, step (b) is performed at room temperature; preferably between 15 and 35° C.; more preferably between 2° and 30° C.; even more preferably at about 25 degrees.

In a particular embodiment, step (b) is performed at atmospheric pressure (i.e. 1 atm).

In a particular embodiment, step (b) is performed under stirring; preferably under a stirring rate of 200 rpm.

### Step (ii)

In a particular embodiment, in step (ii) the precursor compound is added to the solution of step (i) to obtain a second solution.

In a particular embodiment, step (ii) is performed at room temperature; preferably between 15 and 35° C.; more preferably between 2° and 30° C.; even more preferably at about 25 degrees.

In a particular embodiment, step (ii) is performed at atmospheric pressure (i.e. 1 atm).

In a particular embodiment, step (ii) is performed under stirring; preferably under a stirring rate of 200 rpm.

In a particular embodiment, the precursor compound is added to the solution of step (i) to obtain a second solution under stirring.

In a particular embodiment, the molar ratio between the precursor compound added in an step and the AQCs of the solution of previous step are in the range of between 0.1 to 10 equivalents; preferably in the range of between 0.2 and 9 equivalents; more preferably between 0.5 and 8; even more preferably between 0.8 and 7; even much more preferably between 0.9 and 6; more preferably about 1, 1.5, 2, 2.5, 3, 3.5, 4, 4.5, 5, 5.5 or 6 equivalents.

In a particular embodiment, the molar ratio between the precursor compound added in an step number (n) and the AQCs of the solution of the step number (n-1) are in the range of between 0.1 to 10; preferably in the range of between 0.2 and 9; more preferably between 0.5 and 8; even more preferably between 0.8 and 7; even much more preferably between 0.9 and 6; more preferably about 1, 1.5, 2, 2.5, 3, 3.5, 4, 4.5, 5, 5.5 or 6; wherein “n” is an integer.

In a more particular embodiment, the molar ratio between the precursor compound added in a step and the AQCs of the solution of the previous step are in the range of between 0.5 and 2; preferably about 1 equivalent.

Step (iii)

In a particular embodiment, step (iii) comprises repeating step (i) and step (ii) at least once, preferably at least twice; more preferably at least three times; more preferably at least 5 times.

In a particular embodiment, step (iii) consists of repeating step (i) and step (ii) is repeated at least once, preferably at least twice; more preferably at least three times.

In a particular embodiment, the applied current is increased at least in a 0.5%; preferably at least in a 1%; more preferably in at least a 5% in each repetition of step (i).

In a particular embodiment, step (iii) consists of repeating step (ii) at least once, preferably at least twice; more preferably at least three times; even more preferably at least 5 times.

In another particular embodiment, step (iii) comprises repeating step (i) and step (ii) between 2 and 100 times; preferably between 3 and 50 times; more preferably between 4 and 10 times.

In an embodiment, the molar ratio between the precursor added in each step and the AQCs of the solution of previous step is in the range of between 0.1 to 10 equivalents; preferably in the range of between 0.2 and 9; more preferably between 0.5 and 8; even more preferably between 0.8 and 7; even much more preferably between 0.9 and 6; more preferably about 1, 1.5, 2, 2.5, 3, 3.5, 4, 4.5, 5, 5.5 or 6.

In a particular embodiment, the process comprises the following steps:
i. providing
   a metal electrode; wherein the metal of the metal electrode is selected from Ag, Cu, Co, Au, Pt, Fe, Pd and Ni or their bi-metal and multi-metal combinations; wherein the metal electrode is part of a cell further comprising a working and a reference electrode; and
   a polar solvent solution;
   wherein the metal electrode is in contact with the solution;
ii. applying an electric current to the metal electrode of step (i) for between 100 and 1000 s; wherein the electric current is obtained by applying an electrical potential difference between the working and the reference electrode of between 0.1 and 15 V to obtain a solution comprising a polar solvent comprising Atomic Quantum Clusters (AQCs) of formula (II)

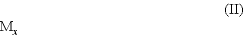

wherein M is at least an element selected from Ag, Cu, Co, Au, Pt, Fe, Pd, Ni or their bi-metal and multi-metal combinations; and wherein x is an integer selected from 2, 3, 4, 5, 6, 7, 8, 9 and 10;
iii. adding a precursor compound to the water solution; wherein the precursor compound comprises at least one of Si or Ti; preferably comprises O and at least one of Si or Ti;
iv. repeating the sequence of steps (ii) and (iii) at least twice; more preferably at least three times;
   wherein the molar ratio between the precursor compound added in each step and the AQCs of the solution of previous step is in the range of between 0.1 to 10 equivalents; preferably between 0.5 and 8 equivalents.

In a particular embodiment, the molar ratio between the precursor compound added in each step and the AQCs of the solution of previous step is constant, preferably is kept constant in each repetition.

In a particular embodiment, the applied current is increased at least in a 0.5%; preferably at least in a 1%; more preferably in at least a 5% in each repetition of step (i).

According to the authors, the process for producing a compound of formula (I) of the present invention is a simple and inexpensive procedure, thus, it can be applied for large-scale production of the compound of formula (I).

An aspect of the present invention is directed to a composition comprising the compound of formula (I) as defined in any of claims 1-6 or the anion of claim 7, and an additional agent or compound; preferably an additional therapeutic agent or compound.

### First Medical Use

In a further aspect, the present invention relates to a compound of formula (I) or an anion as defined in any of the embodiments described above, for use as a medicament.

The above aspect can be formulated as the use of a compound of formula (I) or an anion as defined in any of the embodiments described above, in the manufacture of a medicament.

The above aspect can be formulated as a method of treating or preventing a disease, the method comprising the administration of a compound of formula (I) or an anion as defined in any of the embodiments described above, to a patient in need of such prevention or treatment; preferably the administration of a therapeutically effective amount of said compound of formula (i) or said anion.

In addition, a further aspect is directed to a composition comprising a compound of formula (I) or an anion as defined in any of the embodiments described above, for use as a medicament.

The above aspect can be formulated as the use of a composition comprising a compound of formula (I) or an anion as defined in any of the embodiments described above, in the manufacture of a medicament.

The above aspect can be formulated as a method of treating or preventing a disease, the method comprising the administration of a composition comprising a compound of formula (I) or an anion as defined in any of the embodiments described above, to a patient in need of such prevention or treatment; preferably the administration of a therapeutically effective amount of said compound of formula (i) or said anion.

### Second Medical Uses

In an aspect, the present invention relates to a compound of formula (I) or an anion as defined in any of the embodiments described above, for use in the treatment or prevention of a cell proliferative disorder.

The above aspect can be formulated as the use of a compound of formula (I) or an anion as defined in any of the embodiments described above, in the manufacture of a medicament for the prevention or treatment of cell proliferative disorder such as a tumor and/or cancer.

The above aspect can be formulated as the use of a compound of formula (I) or the anion as defined in any of the embodiments described above, as a medicament for the prevention or treatment of cell proliferative disorder such as a tumor and/or cancer.

The above aspect can be formulated as a method of treating or preventing cell proliferative disorder such as a tumor and/or cancer, the method comprising the administration of a compound of formula (I) or the anion as defined in any of the embodiments described above to a patient in need of such prevention or treatment.

In another aspect, the present invention relates to a composition comprising a compound of formula (I) or the anion as defined in any of the embodiments described above, for use in the treatment or prevention of a cell proliferative disorder such as a tumor and/or cancer.

The above aspect can be formulated as the use of a composition comprising a compound of formula (I) or an anion as defined in any of the embodiments described above, in the manufacture of a medicament for the prevention or treatment of cell proliferative disorder such as a tumor and/or cancer.

The above aspect can be formulated as the use of a composition comprising a compound of formula (I) or ane anion as defined in any of the embodiments described above, as a medicament for the prevention or treatment of cell proliferative disorder such as a tumor and/or cancer.

The above aspect can be formulated as a method of treating or preventing cell proliferative disorder such as a tumor and/or cancer, the method comprising the administration of a compound of formula (I), or of a composition comprising a compound of formula (I) or an anion as defined in any of the embodiments described above to a patient in need of such prevention or treatment.

References to a “cell proliferative disorder” refer to a disorder resulting in the new, abnormal growth of cells or a growth of abnormal cells without physiological control. This can result in an unstructured mass, i.e. a tumour.

In one embodiment, the cell proliferative disorder is a tumour and/or cancer.

A compound of formula (I), an anion or a composition comprising said compound or anion as defined in any of the embodiments described above, may be used to treat cell proliferative disorders including, but not limited to, primary tumours, metastases, precancerous conditions (pre-cancer stages), endometriosis and polycystic ovary syndrome.

Excessive proliferation of cells and turnover of cellular matrix contribute significantly to the pathogenesis of several diseases, including cancer, atherosclerosis, rheumatoid arthritis, psoriasis, idiopathic pulmonary fibrosis, scleroderma and cirrhosis of the liver among others.

Cancers may include, but are not limited to: spleen, colorectal and/or colon cancer, colon carcinomas, ovarian carcinomas, ovarian cancer, endometrial cancer, breast cancer, carcinomas of the uterus, lung cancer, stomach cancer, oesophageal cancer, liver cancer, carcinomas of the pancreas, kidney cancer, bladder cancer, prostate cancer, testicular cancer, bone cancer, thyroid cancer, skin cancer such as melanoma, sarcoma, Kaposi sarcomas, brain cancers such as glioma, medulloblastoma or neuroblastomas, blood cancers such as lymphomas and leukaemias, myosarcomas and head and neck carcinoma. In one embodiment, the cancer is selected from lung, breast, colon or brain cancer (in particular glioblastoma). In a further embodiment, the cancer is brain cancer, in particular brain cancer selected from glioma (such as glioblastoma multiforme, oligodendroglioma, ependymomas, brain stem glioma), craniopharyngioma, haemangioblastoma, malignant meningioma, pineal region tumours and vestibular schwannoma. In a yet further embodiment, the brain cancer is glioma, in particular glioblastoma.

In another embodiment the cell proliferative disorder is selected from the group consisting of atherosclerosis, rheumatoid arthritis, psoriasis, idiopathic pulmonary fibrosis, scleroderma, endometriosis, polycystic ovary syndrome and cirrhosis of the liver; preferably atherosclerosis, rheumatoid arthritis, psoriasis, idiopathic pulmonary fibrosis, scleroderma and cirrhosis of the liver.

In a particular embodiment, the cancer is selected from pancreatic, colorectal, blood cancers such as lymphomas and leukaemias, lung, skin, endometrial, thyroid, stomach, bladder, head and neck, colon, brain or breast cancer; preferably from lung, stomach, or brain cancer, in particular glioblastoma.

The present invention has particular use in the treatment of cancers/tumours with a RAS mutation, such as a KRAS, NRAS or HRAS mutation, in particular KRAS mutations. Such mutations have been shown to cause oxidative stress in the tumour cells which results in high levels of ROS, for example see Shaw et al. (2011) PNAS 108(21): 8773-8778.

Therefore, in one embodiment, the cell proliferative disorder (e.g. cancer and/or tumour) comprises a RAS mutation. In a further embodiment, the RAS mutation is selected from a KRAS, NRAS or HRAS mutation, in particular a KRAS mutation. It will be understood that such cancers/tumours may also be referred to as a RAS mutant cancer, e.g. a KRAS, HRAS or NRAS mutant cancer or tumour. In a yet further embodiment, the RAS mutation is an activating mutation, i.e. the mutation causes increased or constitutive activity of a RAS protein.

The RAS family of proteins are GTPases which hydrolyse GTP to GOP allowing for activation of a number of downstream signaling pathways. For example, KRAS has been shown to be involved in the mitogen activated kinase pathway. Common mutations in KRAS reduce its intrinsic GTPase function, preventing hydrolysis of GTP to GOP, thus locking KRAS in its active state. This results in constitutive activation of downstream signaling pathways that can drive oncogenesis.

Many RAS mutations are known in the art and KRAS mutations are the most frequent oncogenic mutations in human cancer. A cancer comprises a RAS mutation if one or more of the cells in the cancer comprise(s) a RAS mutation. Subjects having RAS mutations may be identified by methods known in the art such as PCR, nucleic acid sequencing, allele-specific PCR methods, single-strand conformational polymorphism analysis, melt-curve analysis, probe hybridization, pyrosequencing (i.e. nucleotide extension sequencing), genotyping, and other sequencing methods (e.g. see Anderson (2011) Expert Rev Mol Diagn. 11 (6): 635-642 and Ogino et al. (2005) J. Mol. Diagn. 7: 413-421).

As shown herein, compound of formula (I) that comprises AQCs and inorganic ligands had a toxic effect on the A549 cell line, which has been shown to comprise a KRAS mutation (such as KRAS G12S where the glycine residue at position 12 is mutated). Furthermore, cells comprising a HRAS mutation (HRasV12 where a mutation of the valine residue at position 12 was mutated) were more sensitive to the toxic effects of the compound of formula (I) that comprises AQCs and inorganic ligands compared to control cells.

It is estimated that 30% of all human cancers carry a RAS mutation. For example, 88% of pancreatic ductal adenocarcinomas, 52% of colorectal cancers, 43% of multiple myelomas, 32% of lung adenocarcinomas, 28% of melanomas, 25% of endometrial cancers, 13% of thyroid cancers, 12% of stomach cancers, 11% of acute myelogenous leukemias, 11% of bladder cancers, 6% of head and neck squamous cell carcinomas and 2% of breast cancers are believed to carry RAS mutations (data compiled from Cancer Cell Line Encyclopedia (CCLE); the International Cancer Genome Consortium (ICGC); and The Cancer Genome Atlas Data Portal (TCGA)).

Therefore, in one embodiment, the cell proliferative disorder (in particular the cell proliferative disorder with a RAS mutation) is selected from pancreatic, colorectal, blood, lung, skin, endometrial, thyroid, stomach, bladder, head and neck or breast cancer. In a further embodiment, the cell proliferative disorder (in particular the cell proliferative disorder with a RAS mutation) is selected from pancreatic, colorectal, blood, lung, skin, endometrial, thyroid, stomach, bladder or head and neck cancer; preferably lung cancer.

In one embodiment, the cell proliferative disorder is pancreatic cancer, e.g. pancreatic ductal adenocarcinoma, particularly a RAS mutant pancreatic cancer, e.g. a RAS mutant pancreatic ductal adenocarcinoma. In an alternative embodiment, the cell proliferative disorder is colorectal cancer, particularly a RAS mutant colorectal cancer. In an alternative embodiment, the cell proliferative disorder is blood cancer, e.g. multiple myeloma or acute myelogenous leukemia, particularly a RAS mutant blood cancer, e.g. a RAS mutant multiple myeloma or RAS mutant acute myelogenous leukemia. In an alternative embodiment, the cell proliferative disorder is lung cancer, e.g. non-small lung cell cancer such as lung adenocarcinoma, particularly a RAS mutant lung cancer, e.g. a RAS mutant non-small cell lung cancer, such as a RAS mutant lung adenocarcinoma. In an alternative embodiment, the cell proliferative disorder is skin cancer, e.g. melanoma, in particular a RAS mutant skin cancer, e.g. a RAS mutant melanoma.

In an alternative embodiment, the cell proliferative disorder is endometrial cancer, in particular a RAS mutant endometrial cancer. In an alternative embodiment, the cell proliferative disorder is thyroid cancer, in particular a RAS mutant thyroid cancer. In an alternative embodiment, the cell proliferative disorder is stomach cancer, in particular a RAS mutant stomach cancer. In an alternative embodiment, the cell proliferative disorder is bladder cancer, in particular a RAS mutant bladder cancer. In an alternative embodiment, the cell proliferative disorder is head and neck cancer, e.g. head and neck squamous cell carcinoma, in particular a RAS mutant head and neck cancer, e.g. a RAS mutant head and neck squamous cell carcinoma.

The present invention has particular use in the treatment of cancers with low drug accessibility, such as large tumours with a low level of vascularity or brain tumours which are separated from the circulatory system by the bloodbrain-barrier. This is due to the neutral charge and small size of the therapeutic compound of formula (I) that comprises AQCs and inorganic ligands, allowing them to access areas in a tumour or cancer which are not easily accessible to traditional antineoplastic drugs.

Evidence is provided herein which shows the ability of the compound of formula (I) to penetrate into the central hypoxic regions of multicellular tumour spheroids.

Preventing and treating metastasis of cancer is a key part of cancer treatment to prevent secondary cancers and relapse. It has been surprisingly found that the compound of formula (I) has an additional beneficial effect of treating cancer metastases, as well as the primary tumour.

Therefore, according to an aspect of the invention, there is provided a compound of formula (I) as described herein or a composition comprising a compound of formula (I), for use in the prevention and/or treatment of metastases, such as lymph node metastases, in particular to treat and/or prevent lung cancer metastasis. According to another aspect of the invention, there is provided the composition as described herein, for use in the prevention and/or treatment of lymph node metastasis of cancer.

In a further embodiment, the lymph node is a mediastinal node. Said mediastinal nodes are a group of lymph nodes located in the thoracic cavity of the body.

### Combination Therapy

One aspect of the present invention is directed to a composition comprising the compound of formula (I) the anion of the present invention as described in any of its particular embodiments, and an additional agent; preferably an additional therapeutic agent.

The compositions described herein may be used in combination with a compound of formula (I) or an anion of the invention as described in any of its particular embodiments.

It has been found that the compounds of formula (I) enable them to intercalate into DNA and result in chromatin de-compaction. This can therefore be used to increase the susceptibility of treated cells to radiation and improve the effectiveness of radiation therapy.

In one embodiment, the compounds of formula (I) or the anions of the present invention (named as the first agent) are administered simultaneously with an additional agent. In this embodiment, the two agents are administered at the same time or at substantially the same time. They may also be administered by the same route and, optionally, in the same composition. Alternatively, they may be administered by different routes, i.e. separately, but at the same time or at substantially the same time.

In an alternative embodiment, the compositions and the compounds of formula (I) or the anions of the present invention, are administered sequentially. In this embodiment, the two agents are administered at different times so that one of the agents is administered before the second agent. For example, the composition may be administered before or after the compound of formula (I) or the anion of the present invention. They may be administered by the same or different routes.

According to another aspect of the invention, there is provided either a compound of formula (I), an anion of the present invention, or a composition comprising a compound of formula (I) or an anion of the present invention, in combination with radiation therapy for use in the treatment of a cell proliferative disorder.

The present inventors have surprisingly found that the compounds of formula (I) or the anions of the present invention, have a catalytic effect on thiol oxidation resulting in cell demise. Therefore, the compounds of formula (I) or the anions of the present invention, may be used on their own as a cancer therapy and thus in one embodiment, the compositions described herein do not include additional antineoplastic drugs.

In one embodiment, the compositions of the present invention comprising the compounds of formula (I) or the anions of the present invention, may include or be used in combination with additional therapeutic agents. Such agents may be active agents which are used in conjunction with cancer therapy, such as agents used as palliative treatments to ameliorate unwanted side effects. Therefore, in one embodiment, the additional therapeutic agent is an agent used as a palliative treatment. In a further embodiment, the palliative treatment is selected from the group consisting of: antiemetic agents, medication intended to alleviate pain such as opioids, medication used to decrease high blood uric acid levels such as allopurinol or rasburicase, anti-depressants, sedatives, anti-convulsant drugs, laxatives, anti-diarrheal drugs and/or antacids.

In one embodiment, the additional therapeutic agent is not an antineoplastic drug. In an alternative embodiment, the additional therapeutic agent is an antineoplastic agent. In one embodiment, the antineoplastic agent is selected from the group consisting of: alkylating agents (e.g. nitrogen mustard analogues, nitrosoureas, alkyl sulfonates, platinum containing compounds, ethylemines, and imidazotetrazines), cytotoxic antibiotics (e.g. anthracyclines, actinomycins), plant alkaloids and other natural products (e.g. campthotecin derivatives, epipodophyllotoxins, taxanes, and vinca alkaloids), antimetabolites (e.g. cytidine analogues, folic acid analogues, purine analogues, pyrimidine analogues, urea derivatives) and drugs for targeted therapy (e.g. kinase inhibitors, and monoclonal antibodies).

In one embodiment, the compound of formula (I), the anion or the composition of the present invention (as first agent) and the additional therapeutic agent are administered simultaneously. In this embodiment, the two agents are administered at the same time or at substantially the same time. They may also be administered by the same route and, optionally, in the same composition. Alternatively, they may be administered by different routes, i.e. separately, but at the same time or at substantially the same time.

In an alternative embodiment, the composition and additional therapeutic agent are administered sequentially. In this embodiment, the two agents are administered at different times so that one of the agents is administered before the second agent. They may be administered by the same or different routes.

In one embodiment, the composition is administered before the additional therapeutic agent. In an alternative embodiment, the composition is administered after the additional therapeutic agent.

In a further aspect, the present invention relates to the compound of formula (I) or the anion as defined in any of the embodiments described above, for use in the treatment or prevention of atherosclerosis, rheumatoid arthritis, psoriasis, idiopathic pulmonary fibrosis, scleroderma, endometriosis, polycystic ovary syndrome and cirrhosis of the liver; preferably atherosclerosis, rheumatoid arthritis, psoriasis, idiopathic pulmonary fibrosis, scleroderma and cirrhosis of the liver.

In a further aspect, the present invention relates to a composition comprising a compound of formula (I) or an anion as defined in any of the embodiments described above, for use in the treatment or prevention of atherosclerosis, rheumatoid arthritis, psoriasis, idiopathic pulmonary fibrosis, scleroderma, endometriosis, polycystic ovary syndrome and cirrhosis of the liver; preferably atherosclerosis, rheumatoid arthritis, psoriasis, idiopathic pulmonary fibrosis, scleroderma and cirrhosis of the liver.

The above aspect can be formulated as the use of a compound of formula (I) or the anions as defined in any of the embodiments described above, as a medicament for the prevention or treatment of atherosclerosis, rheumatoid arthritis, psoriasis, idiopathic pulmonary fibrosis, scleroderma, endometriosis, polycystic ovary syndrome and cirrhosis of the liver; preferably atherosclerosis, rheumatoid arthritis, psoriasis, idiopathic pulmonary fibrosis, scleroderma and cirrhosis of the liver.

The above aspect can be formulated as the use of a composition comprising the compounds of formula (I) or the anions as defined in any of the embodiments described above, as a medicament for the prevention or treatment of atherosclerosis, rheumatoid arthritis, psoriasis, idiopathic pulmonary fibrosis, scleroderma, endometriosis, polycystic ovary syndrome and cirrhosis of the liver; preferably atherosclerosis, rheumatoid arthritis, psoriasis, idiopathic pulmonary fibrosis, scleroderma and cirrhosis of the liver.

The above aspect can be formulated as the use of the compounds of formula (I) or the anions as defined in any of the embodiments described above, in the manufacture of a medicament for the prevention or treatment of atherosclerosis, rheumatoid arthritis, psoriasis, idiopathic pulmonary fibrosis, scleroderma, endometriosis, polycystic ovary syndrome and cirrhosis of the liver; preferably atherosclerosis, rheumatoid arthritis, psoriasis, idiopathic pulmonary fibrosis, scleroderma and cirrhosis of the liver.

The above aspect can be formulated as the use of a composition comprising a compound of formula (I) or an anion as defined in any of the embodiments described above, in the manufacture of a medicament for the prevention or treatment of atherosclerosis, rheumatoid arthritis, psoriasis, idiopathic pulmonary fibrosis, scleroderma, endometriosis, polycystic ovary syndrome and cirrhosis of the liver; preferably atherosclerosis, rheumatoid arthritis, psoriasis, idiopathic pulmonary fibrosis, scleroderma and cirrhosis of the liver.

The above aspect can be formulated as a method of treating or preventing atherosclerosis, rheumatoid arthritis, psoriasis, idiopathic pulmonary fibrosis, scleroderma, endometriosis, polycystic ovary syndrome and cirrhosis of the liver, the method comprising the administration of a compound of formula (I) or an anion as defined in any of the embodiments described above to a patient in need of such prevention or treatment.

The above aspect can be formulated as a method of treating or preventing atherosclerosis, rheumatoid arthritis, psoriasis, idiopathic pulmonary fibrosis, scleroderma, endometriosis, polycystic ovary syndrome and cirrhosis of the liver; preferably atherosclerosis, rheumatoid arthritis, psoriasis, idiopathic pulmonary fibrosis, scleroderma and cirrhosis of the liver, the method comprising the administration of the a composition comprising the compound of formula (I) or the anion as defined in any of the embodiments described above to a patient in need of such prevention or treatment.

In a further aspect, the present invention relates to a compound of formula (I) or an anion as defined in any of the embodiments described above, for use in the treatment or prevention of infections such as yeast infections and/or fungal infections for example topical and systemic fungal infections.

In a further aspect, the present invention relates to a composition comprising a compound of formula (I) or an anion as defined in any of the embodiments described above, for use in the treatment or prevention of infections such as yeast infections and/or fungal infections for example topical and systemic fungal infections.

The above aspect can be formulated as the use of the compounds of formula (I) or the anions as defined in any of the embodiments described above, in the manufacture of a medicament for the prevention or treatment of infections such as yeast infections and/or fungal infections for example topical and systemic fungal infections.

The above aspect can be formulated as the use of a composition comprising a compound of formula (I) or an anion as defined in any of the embodiments described above, in the manufacture of a medicament for the prevention or treatment of infections such as yeast infections and/or fungal infections for example topical and systemic fungal infections.

The above aspect can be formulated as the use of a compound of formula (I) or an anion as defined in any of the embodiments described above, as a medicament for the prevention or treatment of infections such as yeast infections and/or fungal infections for example topical and systemic fungal infections.

The above aspect can be formulated as the use of a composition comprising a compound of formula (I) or an anion as defined in any of the embodiments described above, as a medicament for the prevention or treatment of infections such as yeast infections and/or fungal infections for example topical and systemic fungal infections.

The above aspect can be formulated as a method of treating or preventing infections such as yeast infections and/or systemic and topical fungal infections, the method comprising the administration of a compound of formula (I) or an anion as defined in any of the embodiments described above to a patient in need of such prevention or treatment.

The above aspect can be formulated as a method of treating or preventing infections such as yeast infections and/or systemic and topical fungal infections, the method comprising the administration of a composition comprising a compound of formula (I) or an anion as defined in any of the embodiments described above to a patient in need of such prevention or treatment.

In one embodiment, the infection is caused by overgrowth of yeast, in which case the compound of formula (I) or the anion according to the present invention are used for “yeast infection”.

In another embodiment, the fungal infection can be a topical infection or a systemic infection.

In a particular embodiment, a topical fungal infection is a fungal infection of the skin and mucosae.

In another embodiment, the fungal infection is an aspergillus fungal infection.

The term “treatment” or “to treat” in the context of this specification means administration of the compound of formula (I) or the anion as defined in any of the embodiments described above to ameliorate or eliminate a disease or disorder or one or more symptoms associated with said disease or disorder. “Treatment” also encompasses ameliorating or eliminating the physiological sequelae of the disease or disorder.

The term “prevention” or “to prevent” in the context of this specification means reducing the risk of acquiring or developing a disease or disorder or one or more symptoms associated with said disease or disorder.

The term “effective amount”, “therapeutically effective amount” or “effective dose” refers to the amount sufficient to elicit the desired pharmacological or therapeutic effects, thus resulting in effective prevention or treatment of the disorder. Prevention of the disorder is manifested by delaying the onset of the symptoms of the disorder to a medically significant extent. Treatment of the disorder is manifested by a decrease in the symptoms associated with the disorder or an amelioration of the reoccurrence of the symptoms of the disorder.

The authors have observed that the compound of formulas (I) comprising AQCs and inorganic ligands such as titanates or silicates show similar mechanism of action regarding treatment and prevention of diseases than, naked AQCs or AQCs comprising organic ligands. The activity of the compounds of formula (I) has been confirmed by the results obtained in the examples included in the present patent application. Nevertheless and interestingly, the compound of formula (I) that comprises AQCs and inorganic ligands is much more stable in solution and more resistant to aggregation and agglomeration than bare or naked AQCs or AQCs with organic ligands. In addition, their method of production lead to compound of formula (I) that comprises AQCs and inorganic ligands having less contamination than similar naked AQCs or AQCs with organic ligands.

### Radiation Therapy

Radiation therapy (also referred to as radiotherapy) uses high doses of radiation to damage cellular DNA and therefore kill cancer cells and shrink tumours. Such therapy may be in the form of an external beam or as internal radiation therapy. The choice of radiation therapy can depend on the type of cancer, size of the tumour, tumour location, as well as other factors, such as the age, general health and medical history of the patient and the other types of cancer treatment used.

Radiation therapy is administered to over 50% of all cancers, worldwide, and is of particular importance in developing and middle-income countries. However, effectiveness of radiation therapy is limited by various factors, in-eluding damage to healthy surrounding tissue, proximity of nearby organs and tumours developing radiation resistance. Therefore, there is a significant unmet need for agents to improve efficacy of radiation therapy.

Application of radiation therapy to cancer cells results in an increased production of ROS. As shown by the evidence provided herein, the effect of the compound of formula (I) or the anion as defined in any of the embodiments described above, is potentiated in the presence of ROS. Therefore, the compound of formula (I) or the anion as defined in any of the embodiments described above, are particularly suited as therapeutic agents which enhance the effectiveness of radiation therapy.

According to an aspect of the invention, there is provided a compound of formula (I), an anion or a composition comprising a compound of formula (I) or an anion as defined in any of the embodiments described above, as described herein, in combination with radiation therapy for use in the treatment of a cell proliferative disorder, such as cancer.

Radiation therapy (also referred to as radiotherapy) uses high doses of radiation to damage cellular DNA and therefore kill cancer cells and shrink tumours. Such therapy may be in the form of an external beam or as internal radiation therapy. The choice of radiation therapy can depend on the type of cancer, size of the tumour, tumour location and well as other factors, such as the age, general health and medical history of the patient and the other types of cancer treatment used.

According to an aspect of the invention, there is provided the use of the compound of formula (I), the anion or a composition comprising the compound of formula (I) as defined in any of the embodiments described above, as a radiotherapy sensitizing agent.

According to another aspect of the invention, there is provided the use of the compounds of formula (I), thes anion or a composition comprising a compound of formula (I) or an anion as defined in any of the embodiments described above, as a radiation therapy sensitizing agent for proliferating cells. It will be understood that the term “radiotherapy sensitizing agent”, also referred to as “radiosensitizers”, refers to a drug which is used to enhance/increase the cytotoxic effect of radiation therapy. A cancer or tumour which is affected by radiation therapy is referred to as “radiosensitive”.

According to another aspect, the invention provides a compound of formula (I), an anion or a composition comprising a compound of formula (I) or an anion as defined in any of the embodiments described above for use as a radiation therapy desensitizing agent for non-proliferating cells.

The compounds of formula (I), the anions or a composition comprising a compound of formula (I) or an anion as defined in any of the embodiments described above, may be used to protect non-proliferating (such as non-dividing) cells from radiation therapy. It will be understood that the term “radiation therapy desensitizing agent”, also referred to as “radiodesensitizers”, refers to a drug which is used to reduce/decrease the cytotoxic effect of radiation therapy.

The compounds of formula (I), the anions or a composition comprising a compound of formula (I) or an anion as defined in any of the embodiments described above are therefore particularly advantageous when used in combination with radiation therapy because they have a dual effect of enhancing the effect of radiation therapy on proliferating cells (i.e. cancer cells) while also protecting non-proliferating cells (i.e. non-diseased cells) from harmful radiation.

References to “proliferation” will be understood by a person skilled in the art. As used herein “proliferating cells” refers to cells undergoing cell proliferation, e.g. cell growth and division. In particular, the invention is used to target cancer cells which have rapid, abnormal and/or uncontrolled cell proliferation. In one embodiment, the proliferating cells are cancer cells, precancer cells, or other abnormal, rapidly dividing cells in a subject. Also, as used herein, “non-proliferating cells” refers to cells which are not undergoing cell proliferation. These cells may also be described as “resting”, “arrested”, “quiescent”, “non-dividing”, “non-cycling” or “Go cells”. In one embodiment, the non-proliferating cells are non-cancerous cells.

Radiation therapy may be in the form of an external beam or as internal radiation therapy. In one embodiment, the radiation therapy comprises external beam irradiation. External beam radiation therapy uses a radiation source that is external to the patient, typically either a radioisotope, such as Cobalt-60 (60Co), Cesium-137 (137Cs), or a high energy x-ray source, such as a linear accelerator machine (LINAC). The external source produces a collimated beam directed into the patient to the tumour site. The adverse effect of irradiating of healthy tissue can be reduced, while maintaining a given dose of radiation in the tumourous tissue, by projecting the external radiation beam into the patient at a variety of “gantry” angles with the beams converging on the tumour site. Examples of external radiation therapy treatment, includes, but is not limited to, conformal radiotherapy, intensity modulated radiotherapy (IMRT), image guided radiotherapy (IGRT), 4-dimensional radiotherapy (40-RT), stereotactic radiotherapy and radiosurgery, proton therapy, electron beam radiotherapy, and adaptive radiotherapy. In an alternative embodiment, the radiation therapy comprises internal radiation therapy. In this embodiment, a radiopharmaceutical agent is administered to a patient and placed in the area to be treated. In one embodiment, the radiopharmaceutical agent comprises a radiation-emitting radioisotope. The radioisotopes are well known to a person skilled in the art and may comprise a metallic or non-metallic radioisotope. Suitable metallic radioisotopes include, but are not limited to: Actinium-225, Antimony-124, Antimony-125, Arsenic-?4, Barium-1 03, Barium-140, Beryllium-?, Bismuth-206, Bismuth-207, Bismuth212, Bismuth213, Cadmium-1 09, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-60, Copper-62, Copper-64, Copper-67, Erbium-169, Europium-152, Gallium-64, Gallium-67, Gallium-68, Gadolinium153, Gadolinium-157 Gold-195, Gold-199, Hafnium-175, Hafnium-175-181, Holmium-166, Indium-110, Indium-111, Iridium-192, Iron 55, Iron-59, Krypton85, Lead-203, Lead-21 0, Lutetium-177, Manganese-54, Mercury-197, Mercury203, Molybdenum-99, Neodymium-147, Neptunium-237, Nickel-63, Niobium-95, Osmium-185+191 Palladium-103, Palladium-109, Platinum-195m, Praseodymium-143, Promethium-147, Promethium-149, Protactinium-233, Radium-226, Rhenium-186, Rhenium-188, Rubidium-86, Ruthenium-97, Ruthenium-1 03, Ruthenium-1 05, Ruthenium-1 06, Samarium-153, Scandium-44, Scandium-46, Scandium-4 7, Selenium-75, Silver-10m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yt-40 trium-91, Zinc-65, Zirconium-89, and Zirconium-95. Suitable non-metallic radioisotopes include, but are not limited to: Iodine-131, Iodine-125, Iodine-123, Phosphorus-32, Astatine-211, Fluorine-18, Carbon-11, Oxygen-15, Bromine-76, and Nitrogen-13.

The type of radiation that is suitable for use in the present invention can vary. In one embodiment, the radiation therapy comprises electromagnetic radiation or particulate radiation. Electromagnetic radiation includes, but is not limited to, x-rays and gamma rays. Particulate radiation includes, but is not limited to, electron beams (beta particles), alpha particles, proton beams, neutron beams and negative pi mesons.

In one embodiment, the radiation therapy comprises brachytherapy. In brachytherapy, radiation sources are placed directly at the site of the cancer or tumour. This has the advantage that the irradiation only affects a very localized area thereby minimising exposure to radiation of healthy tissues. Furthermore, this allows the tumour to be treated with very high doses of localized radiation, whilst reducing the probability of unnecessary damage to surrounding healthy tissues.

In one embodiment, the brachytherapy comprises intracavitary treatment or interstitial treatment. Intracavitary treatment comprises placing containers that hold radiation sources into body cavities where the tumour is present or near to where the tumour is present. Interstitial treatment comprises placing containers that hold radioactive sources 55 directly into a tumour or body tissue. These radioactive sources can stay in the patient permanently. Most often, the radioactive sources are removed from the patient after several days. Containers may comprise needles, seeds, wires, or catheters.

In one embodiment, the radiation therapy comprises systemic radioisotope therapy. In systemic radioisotope therapy, radiopharmaceutical agents comprising radioisotopes are delivered through infusion or ingestion. The administered radioisotopes may be targeted due to the chemical properties of the isotope, for example radioiodine which is preferentially absorbed by the thyroid gland. Targeting can also be achieved by conjugating the radioisotope to a targeting moiety, such as a molecule or antibody which binds to the target tissue. In one embodiment, the radiopharmaceutical agent comprises a radioactive conjugate. In a further embodiment, the radioactive conjugate is a radiolabelled antibody.

In one embodiment, the radiopharmaceutical agent is administered orally, parenterally, intraperitoneally, intravenously, intraarterially, transdermally, sublingually, intramuscularly, rectally, transbuccally, intranasally, via inhalation, vaginally, intra-occularly, locally, subcutaneously, intra-adiposally, intraarticularly or intrathecally. In one embodiment, the radiopharmaceutical agent is in a slow release dosage form.

The choice of radiation therapy can depend on the type of cancer, size of the tumour, tumour location and other factors, such as the age, general health and medical history of the patient and the other types of cancer treatment used.

In one embodiment, the composition and radiation therapy are applied simultaneously. In an alternative embodiment, the composition and radiation therapy are applied sequentially, preferably wherein the composition is applied prior to the radiation therapy. If the agents are administered separately, the radiation therapy may be administered while the composition is still effective, i.e. the composition and the radiation therapy are administered within a timeframe that will exert a synergistic or at least a combined effect upon administration to a patient. In one embodiment, the composition is administered not more than 6 hours prior to radiation therapy, such as between 1 and 6 hours prior to radiation therapy.

In a further embodiment, the composition is administered about 6 hours, about 5 hours, about 4 hours, about 3 hours, about 2 hours or about 1 hour prior to radiation therapy. In one embodiment, the therapeutic effect of the composition and the radiation therapy is synergistic. In one embodiment, the composition sensitizes cancer cells in the patient to radiation therapy.

In one embodiment, the compositions of the invention are able to improve the efficacy of the radiation therapy at least two-fold, such as three-fold, four-fold, five-fold or above, compared to the efficacy of the radiation therapy for the treatment of the disorder alone.

### Pharmaceutical Compositions

According to an aspect of the invention, there is provided a pharmaceutical composition comprising the compositions as described herein.

The compositions, and combinations where appropriate, may be formulated as a pharmaceutical composition, optionally comprising a pharmaceutically acceptable excipient, diluent or carrier. The carrier, diluent and/or excipient must be “acceptable” in the sense of being compatible with the other ingredients of the composition and not deleterious to the recipient thereof.

Examples of pharmaceutically acceptable carriers can include one or more of water, saline, phosphate buffered saline, dextrose, glycerol, ethanol and the like, as well as combinations thereof. Suitable pharmaceutical carriers, excipients or diluents are described in “Remington's Pharmaceutical Sciences” by E. W. Martin. Pharmaceutically acceptable carriers may further comprise minor amounts of auxiliary substances such as wetting or emulsifying agents, preservatives or buffers, which enhance the shelf life or effectiveness of the compositions of the invention. Pharmaceutical compositions may also include anti-adherents, binders, coatings, disintegrants, flavours, colours, lubricants, sorbents, preservatives, sweeteners, freeze dry excipients (including lyoprotectants) or compression aids.

Pharmaceutical compositions of the invention may be administered in a plurality of pharmaceutical forms of administrations, e.g. solid (such as tablets, pills, capsules, granules etc.) or liquid (such as solutions, suspensions, syrups, ointments, creams, gels or emulsions).

Pharmaceutical compositions of the invention can comprise a therapeutically effective amount. The therapeutically effective amount (i.e. the amount that produces an effect to help heal or cure the disorder to be treated) that may be administered to a subject will depend on multiple factors, such as the disease state, the age, sex, and weight of the individual, and the ability of the pharmaceutical composition to elicit a desired response in the individual. A therapeutically effective amount is also one in which any toxic or detrimental effects of the pharmaceutical composition of the invention, are outweighed by the therapeutically beneficial effects.

In one embodiment, the compound of formula (I) as defined in any of the embodiments described above, are present in an aqueous solution. In a further embodiment, the aqueous solution comprises dissolved oxygen, such as at least 2 times, or at least 3 times, the concentration of the compound of formula (I) present in the mixture.

In one embodiment, the composition is administered (or is formulated for administration) by any suitable mode of delivery, such as intravenously, intraarterially, intracardially, intracutaneously, subcutaneously, transdermally, interperitoneally, intramuscularly, orally, lingually, sublingually, buccally, intrarectally or by enema.

The compositions of the invention may be administered directly to a target site (i.e. the site of the tumour) or systemically (i.e. into the circulatory system). Targeted administration has the advantage of focusing the therapeutic effect of the composition on the cancer or tumour to be treated. Such administration also minimises side-effects. However, the compositions of the invention are also suitable for systemic administration because the mode of action ensures that cellular apoptosis only occurs in cells with a high level of ROS. Levels of ROS are high in proliferating cells, e.g. cancerous cells. However, in normal, non-proliferating cells, levels of ROS are relatively low, therefore the compound of formula (I) will have less of an effect on normal cells which helps to minimise adverse side effects.

In one embodiment, the composition is administered orally, intravenously or subcutaneously. In a further embodiment, the composition is administered orally. The advantage of the compositions of the present invention is that they may be depleted relatively quickly, therefore any side effects can be minimised because the compound of formula (I) do not persist in the body for an extended period.

A topical application is also possible (e.g. for the treatment of melanomas). A particular form of topical application consists of introducing the composition into a carrier system, in particular a drug delivery system, and implanting said carrier system into the cancerous tissues, wherein said carrier system then releases said composition specifically at the site of the cancerous tissue. In this way it is possible to avoid side effects, as may occur in the case of systemic administration, i.e. to reduce the overall strain on the body.

### Uses

According to an aspect of the invention, there is provided the use of the compounds of formula (I) or the anions or a composition comprising a compound of formula (I) or an anion as described herein for the treatment of a cell proliferative disorder.

According to an aspect of the invention, there is provided the use of the composition as described herein to treat and/or prevent metastasis of cancer. In one embodiment, the composition is used to treat and/or prevent lymph node metastasis of cancer. In a further embodiment, the composition is used to treat and/or prevent metastasis of lung cancer.

According to an aspect of the invention, there is provided the use of a compound of formula (I) or an anion or of a composition comprising a compound of formula (I) or an anion as described herein, as a radiation therapy sensitizing agent for proliferating cells.

Said agent may be used for the treatment of a cell proliferative disorder. According to an aspect of the invention, there is provided the use of a compound of formula (I) or an anion or a composition comprising a compound of formula (I) or an anion as described herein, in combination with radiation therapy for the treatment of a cell proliferative disorder.

According to an aspect of the invention, there is provided the use of a compound of formula (I) or an anion or a composition comprising a compound of formula (I) or an anion as described herein, in the manufacture/preparation of a radiation therapy sensitizing agent for proliferating cells.

According to an aspect of the invention, there is provided the use of a compound of formula (I) or ane anion or a composition comprising a compound of formula (I) or an anion as described herein, as a radiation therapy desensitizing agent for non proliferating cells.

According to an aspect of the invention, there is provided a compound of formula (I) or an anion or a composition comprising a compound of formula (I) or an anion thereor as described herein, for the preparation of a pharmaceutical composition for the treatment of a cell proliferative disorder.

According to an aspect of the invention, there is provided the use of a compound of formula (I) or an anion or a composition comprising a compound of formula (I) or an anion as described herein, in the manufacture of a medicament for the treatment of a cell proliferative disorder.

### Methods of Treatment

According to an aspect of the invention, there is provided a method of preventing and/or treating a cell proliferative disorder comprising administering a compound of formula (I) or an anion or a composition comprising a compound of formula (I) or an anion as described herein, to a patient in need thereof.

In one embodiment, said method does not comprise treating the patient with an additional antineoplastic drug.

According to an aspect of the invention, there is provided a method of preventing and/or treating a cell proliferative disorder comprising administering a therapeutically effective amount of a compound of formula (I) or an anion or a composition comprising a compound of formula (I) or an anion as described herein, to a patient in need thereof.

According to an aspect of the invention, there is provided a method of treating a patient with a cell proliferative disorder comprising administering a a compound of formula (I) or ane anion or a composition comprising a compound of formula (I) or an anion as described herein. The embodiments described herein before for the compounds of formula (I) or the anions or a composition comprising a compound of formula (I) or an anion as described herein may be applied to said methods of treatment (e.g. timing and mode of administration, formulation of composition, etc.).

According to an aspect of the invention, there is provided a method of preventing and/or treating metastasis of cancer comprising administering a compound of formula (I) or an anion or a composition comprising a compound of formula (I) or an anion as described herein as described herein. In one embodiment, the method prevents and/or treats lymph node metastasis of cancer. In a further embodiment, the method prevents and/or treats metastasis of lung cancer.

In one embodiment, the methods of treatment described herein additionally comprise treating the patient with radiation therapy, such as after administration of the composition. As described hereinbefore, the compounds of formula (I) or the anions or a composition comprising a compound of formula (I) or an anion as described herein, has particular use as a radiotherapy sensitizing agent.

In one embodiment, the compound of formula (I) or the anion or a composition comprising the compound of formula (I) as described herein, is administered orally, intravenously or subcutaneously.

In one embodiment, the compound of formula (I) or the anion or a composition comprising the compound of formula (I) as described herein is administered simultaneously or prior to the radiation therapy.

The patient may be any subject suffering from the disorder. In one embodiment, the patient is a mammal. In a further embodiment, the mammal is selected from a human or a mouse.

In one embodiment, the therapeutic effect of the composition and the radiation therapy is synergistic. In one embodiment, the compound of formula (I) or the anion or a composition comprising the compound of formula (I) as described herein sensitizes cancer cells in the patient to radiation therapy.

The method comprises administering a therapeutically effective amount of radiation. The amount of radiation used in radiation therapy is measured in Gray (Gy) units and varies depending on the type and stage of cancer being treated. Furthermore, the total dose of radiation may be divided into multiple, smaller doses known as “fractions” over a period of several days in order to minimise the negative side effects. A typical fractionation schedule for adults is 1.8 to 2 Gy per day, five days a week. A typical fractionation schedule for children is 1.5 to 1.8 Gy per day, five days a week.

In one embodiment, a total of at least about 10 Gy, such as 15 Gy, 20 Gy, 25 Gy, 30 Gy, Gy, 40 Gy, 45 Gy, 50 Gy, 55 Gy, 60 Gy, 65 Gy, 70 Gy, 75 Gy, 80 Gy, 85 Gy, 90 Gy, 95 Gy or 100 Gy is administered to a patient in need thereof. The patient may receive radiation three, four or five times a week. An entire course of treatment may last from one to seven weeks depending on the type of cancer and the goal of treatment. In one embodiment, radiation therapy occurs over a period of at least 2, 3 or 4 weeks, such as 2-6 weeks, such as 2-4 weeks, or 5-8 weeks, in particular 5-7 weeks. For example, a patient can receive a dose of 2 Gy/day over about 30 days (i.e. 4-5 weeks).

In one embodiment, the radiation is administered at least once per day for five consecutive days per week. For example, the radiation is administered in at least about 2 Gy fractions at least once per day. In one embodiment, the radiation is administered every other day, three times per week. For example, radiation is administered in 10 Gy fractions every other day, three times per week.

In one embodiment, the radiation therapy is hypofractionated. Hypofractionation is a treatment regimen that delivers higher doses of radiation in fewer visits. In an alternative embodiment, the radiation therapy is hyperfractionated.

Hyperfractionation is a treatment regimen that divides the total dose into more deliveries. It will be appreciated that many other factors are considered when selecting a dose, including whether the patient is receiving chemotherapy, patient comorbidities, whether radiation therapy is being administered before or after surgery, and the degree of success of surgery.

According to another aspect, the invention provides a method of preventing damage to non-proliferating cells in a patient undergoing radiation therapy, comprising administering a therapeutically effective amount of the compound of formula (I) or the anion or a composition comprising the compound of formula (I) as described herein to said patient prior to radiation therapy.

According to an aspect of the invention, there is provided a method of treating metastases, such as lymph node metastases, comprising administering a therapeutically effective amount of the compound of formula (I) or the anion or a composition comprising the compound of formula (I) as described herein, to a patient in need thereof, in combination with radiation therapy.

### Kits

According to an aspect of the invention, there is provided a kit-of-parts comprising: the compound of formula (I) or the anion or a composition comprising the compound of formula (I) as described herein, optionally in admixture with a pharmaceutically acceptable adjuvant, diluent or carrier. The kit according to this aspect of the invention may be used in the treatment of a cell proliferative disorder.

In one embodiment, the kit may be used in combination with radiation therapy for the treatment of a cell proliferative disorder.

### Additional Aspects

According to an aspect of the invention, there is provided an apoptotic agent comprising the compound of formula (I) or the anion or a composition comprising the compound of formula (I) as described herein. The apoptotic agent may comprise the composition as described herein.

According to another aspect of the invention, there is provided a method of inducing thiol oxidation comprising administering the compound of formula (I) or the anion or a composition comprising the compound of formula (I) as described herein, optionally in combination with reactive oxygen species (ROS).

### Other Uses

In a further aspect, the present invention is directed to the use of a compound of formula (I) or an anion or a composition comprising a compound of formula (I) or an anion as described herein, as catalyst; preferably as redox reactions catalyst or as photocatalyst.

The above aspect can be formulated as a catalyst method for a chemical reaction that comprises a step of putting in contact at least one of the reactants of the chemical reaction to be catalysed with a compound of formula (I) or an anion or the composition comprising a compound or an anion as defined in any of the embodiments described above.

Throughout the description and claims the word “comprises” and variations of the word, are not intended to exclude other technical features, additives, components or steps.

Furthermore, the word “comprise” encompasses the case of “consisting of”. Additional objects, advantages and features of the invention will become apparent to those skilled in the art upon examination of the description or may be learned by practice of the invention.

The present invention will be described in further detail with reference to the following examples. These examples are for illustrative purposes only and are not intended to limit the scope of the present invention.

### EXAMPLES

The invention is illustrated by means of the following examples which in no case limit the scope of the invention.

### Example 1: Electrochemical Synthesis of Cu₅-Silicate Atomic Quantum Cluster in Water

Cu5-silicate atomic quantum clusters were synthesized in water as follows.

Two copper electrodes (copper foil glued with Araldite on glass slide, 99.9% Cu, 50×50×50 mm) were polished with a lapping film in wet conditions until obtaining a smooth and completely oxide-free surface.

The electrodes were then put into a beaker with milli-Q water and sonicated for 5 minutes into an ultrasonic bath with temperature not exceeding 30° C. to prevent oxidation of the copper foil. The electrodes were then washed thoroughly with milli-Q water.

The two copper electrodes (at least one acting as working electrode WE) and a Normal hydrogen electrode (NHE) reference electrode were placed in their correspondent places in a glass beaker with a PTFE cap and filled (with stirrer) with 250 mL milli-Q water forming a cell. The beaker was then closed with the cap and put it into a thermostatic water bath at 25° C. Potentiostat wires were then connected to their corresponding electrode and a stirring rate of 200 rpm was set.

Then, the following steps were performed:
1.5V (WE vs. NHE) for 500 seconds was applied. (Intensity range: 0.100-0.500 mA)
31.25 μL of a sodium monosilicate solution in water ([Si]=4 g/L) was added to the beaker for obtaining a concentration of [Si]=0.5 mg/L.
1.5V (WE vs. NHE) were applied for 500 seconds. (Intensity range: 0.500-1 mA)
62.5 μL of a sodium monosilicate solution in water ([Si]=4 g/L) was added to the beaker for obtaining a concentration of [Si]=1.5 mg/L.
1.5V (WE vs. NHE) was applied for 500 seconds. (Intensity range: 1-6 mA)
93.75 μL of a sodium monosilicate solution in water ([Si]=4 g/L) was added to the beaker for obtaining a concentration of [Si]=3 mg/L.
1.5V (WE vs. NHE) was applied for 500 seconds. (Intensity range: 3.5-11 mA)
125 μL of a sodium monosilicate solution was added in water ([Si]=4 g/L) to the beaker for obtaining a concentration of [Si]=5 mg/L.
1.5V (WE vs. NHE) was applied for 500 seconds. (Intensity range: 4-15 mA)
187.5 μL of a sodium monosilicate solution in water ([Si]=4 g/L) was added to the beaker for obtaining a concentration of [Si]=8 mg/L.
1.5V (WE vs. NHE) was applied for 500 seconds. (Intensity range: 8-17 mA)
125 μL of a sodium monosilicate solution in water ([Si]=4 g/L) was added to the beaker for obtaining a final concentration of [Si]=10 mg/L.
1.5V (WE vs. NHE) was applied for 500 seconds. (Intensity range: 10-20 mA)

Then, the wires were disconnected from the cap and the electrodes removed from the beaker. Then the obtained compounds were transferred into a glass bottle for characterization and storage at room temperature, preferably in the absence of light.

Following the previous synthesis method Cu₅-silicate atomic quantum clusters compounds such as Na₂[Cu₅SiO₃] or Na₄[Cu₅(SiO₃)₂], were synthesized in water.

### Example 2

A similar method that the one described in Example 1 was used to obtain five atoms Ag₅-silicate atomic quantum clusters compounds such as [Ag₅SiO₃]²⁻ or [Ag₅(SiO₃)₂]⁴⁻ and at least a counterion (for example a cation such as sodium), in water. Instead of copper electrodes, silver electrodes were used.

The authors of the present invention have observed that AQCs derivatives comprising SiO₃²⁻ or TiO₃²⁻ ligands and at least a counterion (for example a cation such as sodium), were more stable in solution than naked AQCs or AQCs stabilized with organic ligands, and showed reduced or no agglomeration. In addition, AQCs derivatives comprising SiO₃²⁻ or TiO₃²⁻ ligands and at least a counterion are not adsorbed on glass or other vitreous surfaces, thus making easier their study, purification and manipulation.

### Example 3: Efficacy of the Ag5-Silicate Atomic Quantum Clusters Compounds as an Anticancer Agent Against A549 Lung Cancer Cells (a KRAS Mutant Cell Line)

The efficacy as anticancer agents of five atoms Ag₅-silicate atomic quantum clusters compounds with at least a counterion (for example a cation such as sodium) such as Na₂[Ag₅SiO₃] or Na₄ [Ag₅(SiO₃)₂] prepared as described in Example 2, was tested. Please notice that those compounds will be named as Ag₅-silicate atomic quantum clusters compounds to be more concise.

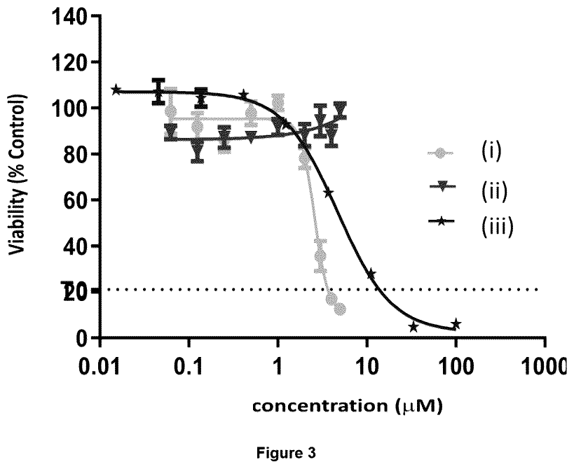

A549 cells were treated for 1 hour with Ag₅-silicate atomic quantum clusters compounds, or for 48 hours with Cisplatin. In addition Ag+ was used as control. The cell viability % was measured 48 hours after the treatment initiation in all cases. FIG. 3 shows the % viability vs dose response (expressed as micro molar concentration) results curve for A549 cells for (i) Ag₅-silicate atomic quantum clusters compounds, (ii) Ag+ as control and (iii) Cisplatin. Results show high cell killing results at low concentration of Ag₅-silicate atomic quantum clusters compounds. A549 cells are adenocarcinomic human alveolar basal epithelial cells, i.e. lung cancer cells.

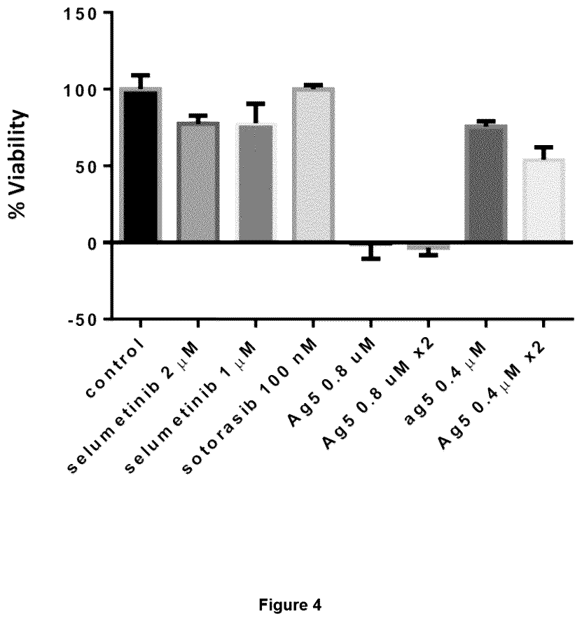

In addition, FIG. 4 shows the % viability response data for A549 cells results for Ag+ used as control, different concentrations of selumetinib, sotorasib, Ag₅-silicate atomic quantum clusters compounds at different concentrations.

Selumetinib is known in the art as an small molecule inhibitor of the mitogen activated protein kinase 1 and 2 (MEK1/2). Sotorasib is known in the art as indicated for the treatment of KRAS G12C mutation-positive non-small cell lung cancer.

As shown herein, Ag₅-silicate atomic quantum clusters compounds had a toxic effect on the A549 cell line, which has been shown to comprise a KRAS mutation (such as KRAS G12S where the glycine residue at position 12 is mutated).

### Example 4: Combinatorial Efficacy of the Ag5-Silicate Atomic Quantum Clusters Compounds with Multiple Targeted Therapies in Several Cancer Cell Lines

The combinatorial efficacy of five atoms Ag₅-silicate atomic quantum clusters compounds such as Na₂[Ag₅SiO₃] or Na₄ [Ag₅(SiO₃)₂] prepared as described in Example 2, with multiple targeted therapies in several cancer cell lines was tested.

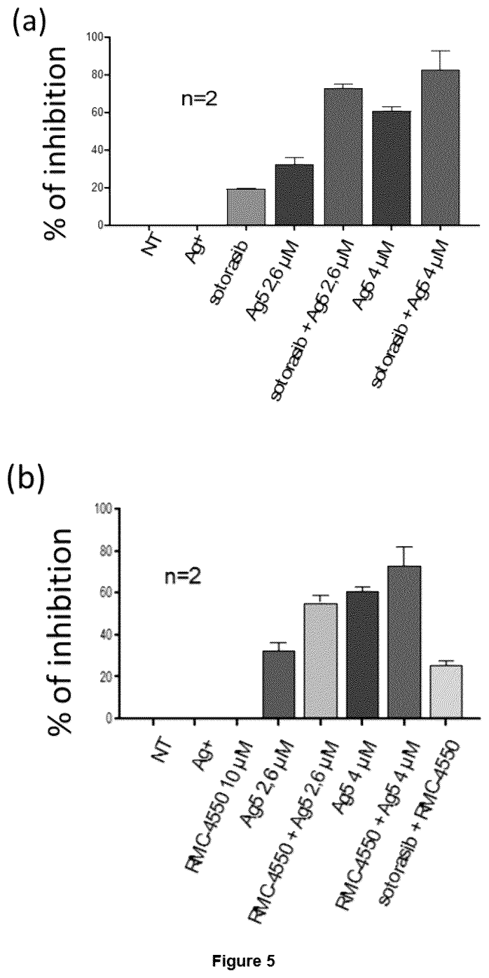

FIG. 5 shows: (a) the % of inhibition results for NCI-H358 cells (lung cancer) non treated, treated with Ag+ as control, Sotorasib (G12C Kras Inhibitor) in an amount of 100 nM for 24 hours, five atoms Ag₅-silicate atomic quantum clusters at different concentrations (2.6 μM and 4 μM) for 1 hour, and a combination of Sotorasib and five atoms Ag₅-silicate atomic quantum clusters at different concentrations; and (b) the % of inhibition results for NCI-H358 cells (lung cancer) treated with Ag+ as control, RMC-4630 (Shp2 inhibitor) in an amount of 10 μM for 24 hours, five atoms Ag₅-silicate atomic quantum clusters at different concentrations (2.6 μM and 4 μM) for 1 hour, a combination of RMC-4630 and five atoms Ag₅-silicate atomic quantum clusters at different concentrations and a combination of sotorasib and RMC-4630. The cell viability was measured after 48 hours of the treatment initiation.

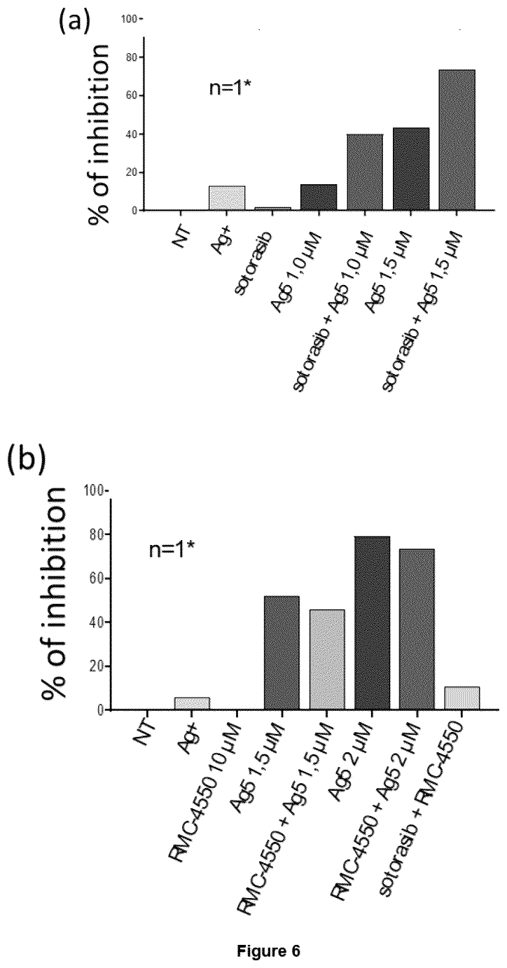

FIG. 6 shows: (a) the % of inhibition results for NCI-H23 cells (lung cancer) non treated, treated with Ag+ as control, Sotorasib (G12C Kras Inhibitor) in an amount of 100 nM for 24 hours, five atoms Ag₅-silicate atomic quantum clusters at different concentrations (1.0 μM and 1.5 μM) for 1 hour, and a combination of Sotorasib and five atoms Ag₅-silicate atomic quantum clusters at different concentrations; and (b) the % of inhibition results for NCI-H23 cells (lung cancer) treated with Ag+ as control, RMC-4630 (Shp2 inhibitor) in an amount of 10 μM for 24 hours, five atoms Ag₅-silicate atomic quantum clusters at different concentrations (1.5 μM and 2 μM) for 1 hour, a combination of RMC-4630 and five atoms Ag₅-silicate atomic quantum clusters at different concentrations and a combination of sotorasib and RMC-4630. The cell viability was measured after 48 hours of the treatment initiation.

Results show that the combinatorial efficacy of five atoms Ag₅-silicate atomic quantum clusters compounds such as Na₂[Ag₅SiO₃] or Na₄ [Ag₅(SiO₃)₂] with multiple targeted therapies in several cancer cell lines gives an additive or greater than additive treatment benefit, which is not obviously predictable. This effect is seen in multiple cell lines with multiple agents such as the mek inhibitor Selumetinib, the SOS1 inhibitor BI-3406, cisplatin, doxorubicin etc.

### Example 5: Ag5-Silicate Atomic Quantum Clusters Compounds Amplify the Therapeutic Effect of External Beam Radiation of Multiple Modalities in Several Cell Types

The therapeutic effect of five atoms Ag₅-silicate atomic quantum clusters compounds such as Na₂[Ag₅SiO₃] or Na₄ [Ag₅(SiO₃)₂] with an external beam radiation was tested in several cell lines. The clonogenic assay of cell viability was used.

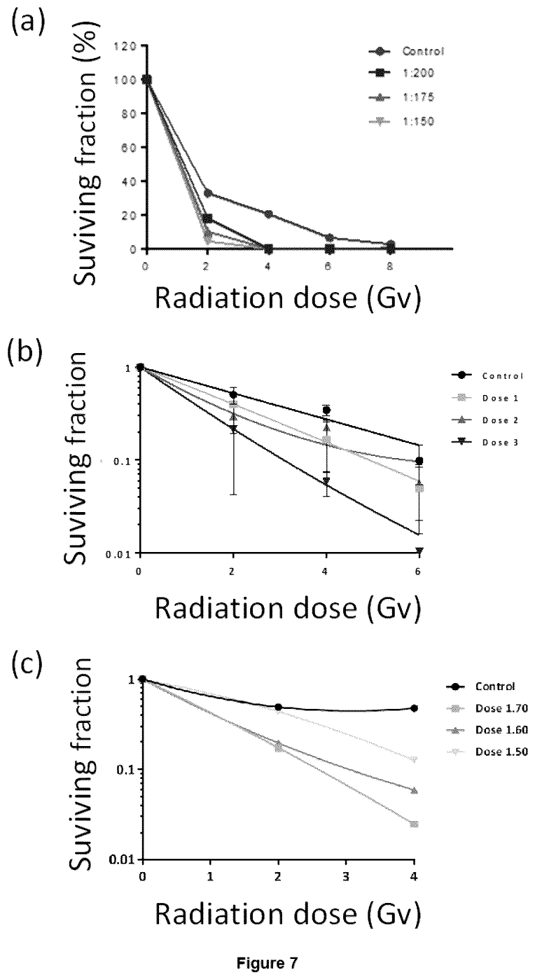

FIG. 7 shows the surviving fraction results vs radiation dose of combining five atoms Ag₅-silicate atomic quantum clusters compounds with an external beam radiation. Results of FIG. 6 showed that Ag5-silicate atomic quantum clusters compounds amplifies the cell killing effect of photon beam radiation radiotherapy in (a) human glioblastoma U251 GBM cell line, (b) human A549 cancer cell line; and (c) U251 cell line. In addition, Ag₅-silicate atomic quantum clusters compounds has the same amplifying effect when co-dosed with proton beam radiation.

### Example 6: In Vivo Efficacy of the Ag5-Silicate Atomic Quantum Clusters Compounds in Orthotopic Lung Cancer Model in Primary Tumor and Metastatic Site (A549 KRASmut G12S, Keap1Mut)

The in vivo efficacy of the Ag5-silicate atomic quantum clusters compounds such as Na₂[Ag₅SiO₃] or Na₄ [Ag₅(SiO₃)₂] in orthotopic lung cancer model in primary tumor and metastatic site (A549 cells are KRASmut, SMARCA4mut, Keap1mut) was tested.

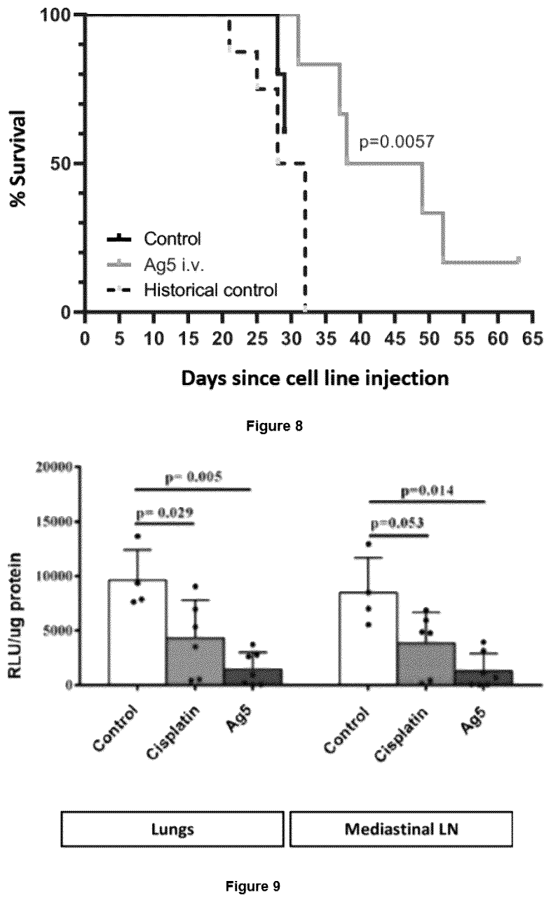

FIG. 8 shows the survival % vs days since the cell line injection results for the control sample (with no treatment), for the historical control and for the sample treated with Ag5-silicate atomic quantum clusters compounds.

FIG. 9 shows the RLU (μg protein) results for the A549-luc cells of the control (no treatment) samples and of the samples treated with Cisplatin (4 mg/kg) and with Ag5-silicate atomic quantum clusters compounds (0.25 mg/kg).

Results showed high efficacy in the treatment with Ag5-silicate atomic quantum clusters compounds in difficult-to-treat orthotopic model, with evidence of enhanced efficacy in metastatic deposits. In addition, MTD was not reached.

### Example 7: Combinatorial Efficacy of the Ag5-Silicate Atomic Quantum Clusters Compounds with Multiple Targeted Therapies in Several Cancer Cell Lines

The combinatorial efficacy of five atoms Ag₅-silicate atomic quantum clusters compounds such as Na₂[Ag₅SiO₃] or Na₄ [Ag₅(SiO₃)₂] prepared as described in Example 2 with multiple targeted therapies in several cancer cell lines was tested.

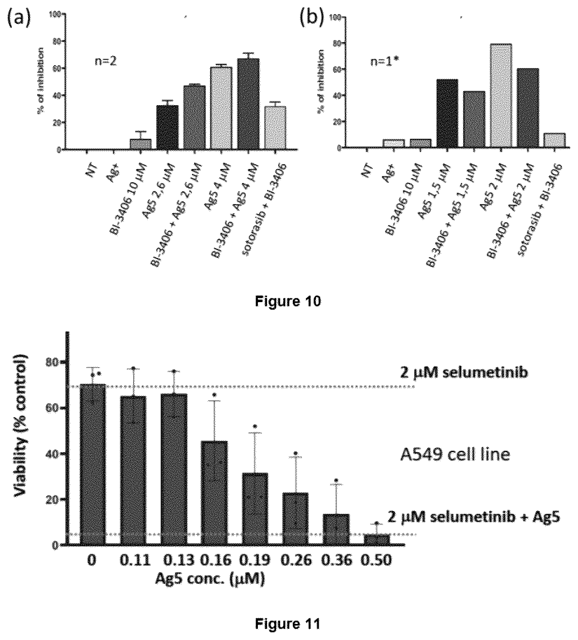

### FIG. 10 Shows:

(a) the % of inhibition results for NCI-H358 cells (lung cancer) non treated, treated with Ag+ as control, treated with BI-3406 (Sos1 inhibitor) in an amount of 10 μM for 24 hours, five atoms Ag₅-silicate atomic quantum clusters at different concentrations (2.6 μM and 4 μM) for 1 hour, a combination of BI-3406 and five atoms Ag₅-silicate atomic quantum clusters at different concentrations and a combination of a combination of BI-3406 and sotorasib; and
(b) the % of inhibition results for NCI-H23 cells (lung cancer) non treated, treated with Ag+ as control, treated with BI-3406 (Sos1 inhibitor) in an amount of 10 μM for 24 hours, five atoms Ag₅-silicate atomic quantum clusters at different concentrations (2.6 μM and 4 μM) for 1 hour, a combination of BI-3406 and five atoms Ag₅-silicate atomic quantum clusters at different concentrations and a combination of a combination of BI-3406 and sotorasib. The cell viability was measured after 48 hours of the treatment initiation.

### Example 8: Ag5-Silicate Atomic Quantum Clusters Compounds Orthogonal Combinations Across Therapeutic Modalities

The Ag5-silicate atomic quantum clusters compounds such as Na₂[Ag₅SiO₃] or Na₄ [Ag₅(SiO₃)₂] orthogonal combinations across therapeutic modalities have been tested.

FIG. 11 shows the viability % vs the Ag5-silicate atomic quantum clusters compounds concentration in micromoles (μM) for a A549 cell line in comparison with the viability results % of 2 μM of selumetinib, AZ and of the combination of 2 μM of selumetinib and Ag5-silicate atomic quantum clusters compounds.

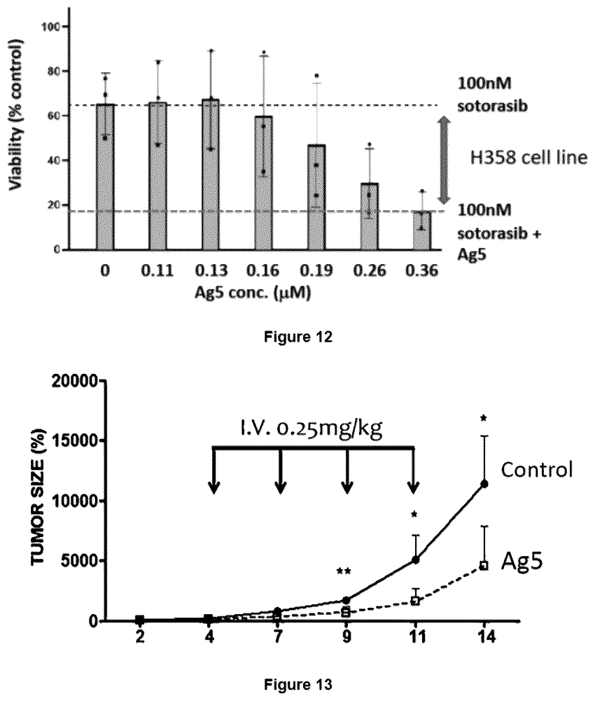

FIG. 12 shows the viability % vs the Ag5-silicate atomic quantum clusters compounds concentration in micromoles (μM) for a H359 cell line in comparison with the viability results % of 100 nM of sotorasib and of the combination of 100 nM of sotorasib and Ag5-silicate atomic quantum clusters compounds.

### Example 8: Ag5-Silicate Atomic Quantum Clusters Compounds are Tested for the Treatment of Glioblastoma Multiforme

Ag5-silicate atomic quantum clusters compounds such as Na₂[Ag₅SiO₃] or Na₄ [Ag₅(SiO₃)₂] have been tested for the treatment of Glioblastoma Multiforme.

FIG. 13 shows the results for tumor size (%) over in vivo monitoring tumor grow for a control sample and a sample treated with Ag5-silicate atomic quantum clusters compounds on a U87 Orthotopic in vivo model.

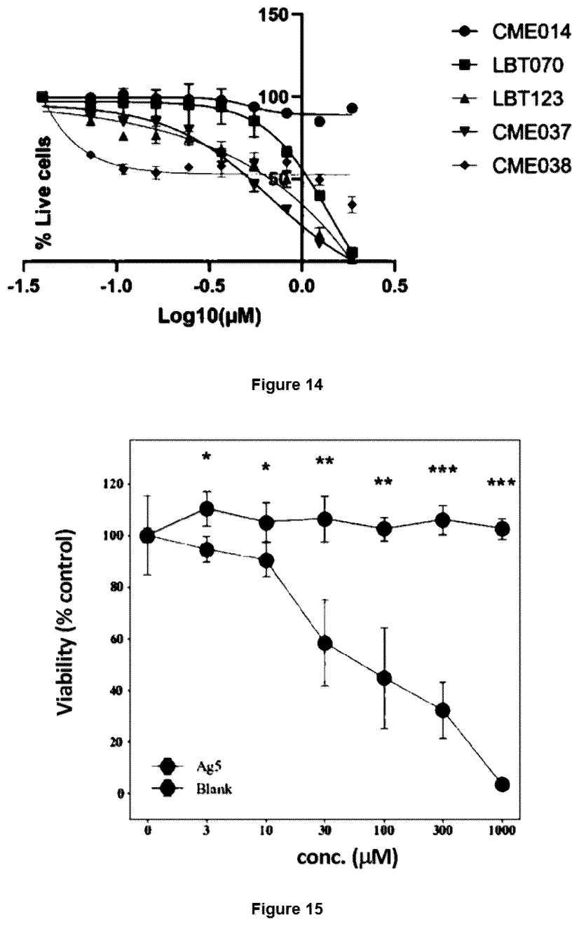

FIG. 14 shows the results of the % of live cells vs the log 10 of μM of Ag5-silicate atomic quantum clusters compounds in an in vitro treatment of a patient derived Glioblastoma Multiforme (GBM) cell lines (20+ lines now tested).

### Example 9: Ag5-Silicate Atomic Quantum Clusters Compounds are Tested for the Treatment of Gastric and Gastro-Oesophageal Carcinoma

Ag5-silicate atomic quantum clusters compounds such as Na₂[Ag₅SiO₃] or Na₄ [Ag₅(SiO₃)₂] have been tested for the treatment of gastric and gastro-oesophageal carcinoma.

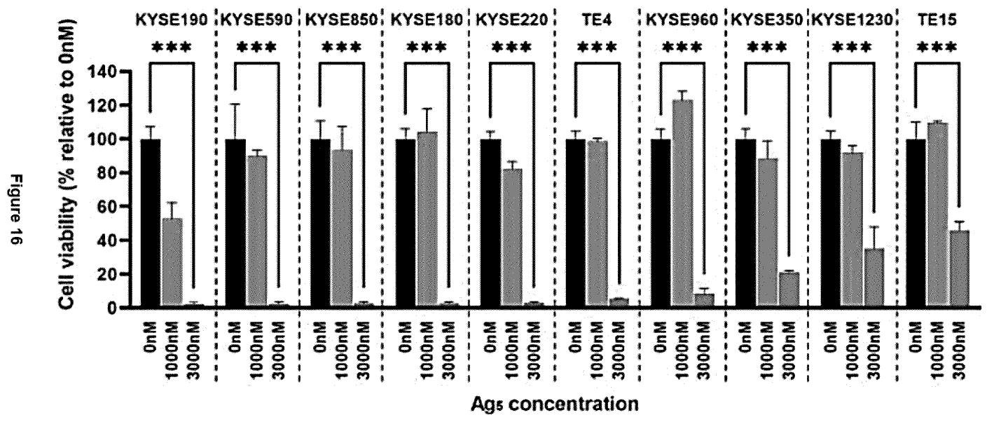

FIG. 15 shows the cell viability % over the Ag5-silicate atomic quantum clusters compounds micro molar concentration (μM) in patient derived esophageal cancer cell line (KYSE350)

FIG. 16 shows the cell viability (%) over the Ag5-silicate atomic quantum clusters compounds micro molar concentration of a 72 h treatment by Dunnett's test.

1. A compound of formula (I) 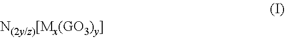
wherein:
N is at least a cation with one or two positive charges; and
z is 1 or 2; and
wherein [Mₓ(GO₃)_{y}] is an anion wherein
Mₓ is an Atomic Quantum Cluster (AQC) consisting of x number of zero valent metal atoms; optionally wherein M is a zero valent metal element selected from Ag, Cu, Co, Au, Pt, Fe, Pd, Ni or their bi-metal and multi-metal combinations and wherein x is an integer selected from 2, 3, 4, 5, 6, 7, 8, 9 and 10; and
G is Si, Ti or a combination thereof; and
y is an integer selected from 1, 2, 3, 4, 5 and 6; and
wherein the compound of formula (I) has no net charge.

2. A compound of formula (I) according to claim 1,
wherein M is selected from Ag, Cu, Au and Pt or their bi-metal and multi-metal combinations; or
wherein x is an integer selected from 3, 4, 5, 6, 7, and 8; or
wherein v is 1 or 2; or
wherein N is an alkali metal cation, an alkaline earth metal cation or combinations thereof.

3. (canceled)

4. (canceled)

5. (canceled)

6. A compound of formula (I) according to claim 1, wherein
N is an alkali metal cation or an alkaline earth metal cation;
z is 1 or 2;
Mₓ is an Atomic Quantum Cluster (AQC) consisting of x number of zero valent metal atoms; wherein M is selected from Ag, Pt, Cu and Au; and wherein x is an integer selected from 3, 4, 5, 6, 7, 8, 9 and 10;
G is Si or Ti; and
y is an integer selected from 1, 2, 3, 4, 5 and 6.

7. An anion consisting of
Atomic Quantum Clusters (AQCs) consisting of 2, 3, 4, 5, 6, 7, 8, 9 and 10 zero valent metal atoms; wherein the metal atoms are selected from Ag, Co, Cu, Au, Pt, Fe, Pd, Ni or their bi-metal and multi-metal combinations; and
an “y” number of anionic ligands of formula (GO₃²⁻) wherein G is Si or Ti;
wherein y is an integer selected from 1, 2, 3, 4, 5 and 6.

8. A process for producing a compound of formula (I) as defined in claim 1, comprising the following steps:
i. providing:
a first solution comprising:
a polar solvent, and
Atomic Quantum Clusters (AQCs) of formula (II) 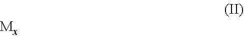
wherein Mₓ is an Atomic Quantum Cluster (AQC) consisting of x number of zero valent metal atoms; optionally wherein M is selected from Ag, Cu, Co, Au, Pt, Fe, Pd, Ni or their bi-metal and multi-metal combinations; and wherein x is an integer selected from 2, 3, 4, 5, 6, 7, 8, 9 and 10; and
a precursor compound; wherein the precursor compound comprises at least one of Si or Ti;
ii. adding the precursor compound to the solution of step (i) to obtain a second solution;
iii. optionally repeating step (i) and/or (ii); and
wherein the molar ratio between the precursor compound added in each step and the AQCs of the solution of previous step are in the range of between 0.1 to 10 equivalents.

9. The process according to claim 8, wherein the solution of step (i) is obtained by a process comprising the following steps:
a. providing
a metal electrode; optionally wherein the metal is selected from Ag, Cu, Co, Au, Pt, Fe, Pd, Ni or their bi-metal and multi-metal combinations; and
a solution comprising a polar solvent;
wherein the metal electrode is in contact with the solution;
b. applying an electric current for at least 50 s to the electrode of step (a) to obtain a solution comprising a polar solvent and Atomic Quantum Clusters (AQCs) of formula (II) 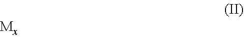
wherein Mₓ is an Atomic Quantum Cluster (AQC) consisting of x number of zero valent metal atoms; optionally, wherein M is selected from Ag, Co, Cu, Au, Pt, Fe, Pd, Ni or their bi-metal and multi-metal combinations; and wherein x is an integer selected from 2, 3, 4, 5, 6, 7, 8, 9 and 10.

10. The process according to claim 9, wherein the metal electrode of step (a) is part of a cell further comprising a working electrode and a reference electrode; and wherein the electric current of step (b) is obtained by applying an electrical potential difference between the working and the reference electrode.

11. The process according to claim 10, wherein the electrical potential difference is between 0.1 and 15 V.

12. The process according to claim 8,
wherein the polar solvent is selected from water, acetonitrile, chloroform, dichloromethane, acetic acid, ethanol, methanol, isopropyl alcohol, and mixtures thereof; or
wherein the process comprises the following steps:
i. providing
a metal electrode; wherein the metal of the metal electrode is selected from Ag, Cu, Co, Au, Pt, Fe, Pd and Ni or their bi-metal and multi-metal combinations; wherein the metal electrode is part of a cell further comprising a working and a reference electrode; and
a polar solvent solution;
wherein the metal electrode is in contact with the polar solvent solution;
ii. applying an electric current to the metal electrode of step (i) for between 100 and 1000
s: wherein the electric current is obtained by applying a electrical potential difference between the working and the reference electrode of between 0.1 and 15 V to obtain a solution comprising a polar solvent comprising Atomic Quantum Clusters (AOCs) of formula (II) 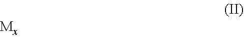
wherein M is at least an element selected from Ag, Cu, Co, Au, Pt, Fe, Pd, Ni or their bi-metal and multi-metal combinations; and wherein x is an integer selected from 2, 3, 4, 5, 6, 7, 8, 9 and 10;
iii. adding a precursor compound to the polar solvent solution; wherein the precursor compound comprises at least one of Si or Ti;
iv. repeating steps (ii) and/or (iii) at least twice;
wherein the molar ratio between the precursor compound added in each step and the AQCs of the solution of previous step is in the range of between 0.1 to 10 equivalents.

13. (canceled)

14. A composition comprising a compound of formula (I) as defined in claim 1, and an additional compound or agent.

15. A catalyst method for a chemical reaction that comprises a step of putting in contact at least one of the reactants of the chemical reaction with the compound of formula (I) as defined in claim 1 or the anion of said compound, or a composition comprising said compound and an additional compound or agent, as catalyst.

16. (canceled)

17. A method of treating and/or preventing a cell proliferative disorder, the method comprising the administration of the compound of formula (I) as defined in claim 1 or the anion of said compound, or a composition comprising said compound and an additional compound or agent, to a patient in need of such treatment and/or prevention.

18. A method of treating and/or preventing a tumor, the method comprising the administration of the compound of formula (I) as defined in claim 1 or the anion of said compound, or a composition comprising said compound and an additional compound or agent, to a patient in need of such treatment and/or prevention.

19. A method of treating and/or preventing cancer, the method comprising the administration of the compound of formula (I) as defined in claim 1 or the anion of said compound, or a composition comprising said compound and an additional compound or agent, to a patient in need of such treatment and/or prevention.

20. A method of treating and/or preventing a spleen, pancreatic, colorectal and/or colon cancer, colon carcinomas, ovarian carcinomas, ovarian cancer, endometrial cancer, breast cancer, carcinomas of the uterus, lung cancer, stomach cancer, oesophageal cancer, liver cancer, carcinomas of the pancreas, kidney cancer, bladder cancer, prostate cancer, testicular cancer, bone cancer, thyroid cancer, skin cancer, sarcoma, Kaposi sarcomas, brain cancers, blood cancers, myosarcomas and head and neck carcinoma, the method comprising the administration of the compound of formula (I) as defined in claim 1 or the anion of said compound, or a composition comprising said compound and an additional compound or agent, to a patient in need of such treatment and/or prevention.

21. The method of treating and/or preventing according to claim 20, wherein the cancer is selected from pancreatic, colorectal and/or colon cancer, colon carcinomas, blood cancers, lung, skin, endometrial, thyroid, stomach, bladder, head and neck, colon, brain cancers, or breast cancer.

22. The method of treating and/or preventing according to claim 21, wherein the cancer is selected from lung, stomach, or brain cancers.

23. A method of treating and/or preventing atherosclerosis, rheumatoid arthritis, psoriasis, idiopathic pulmonary fibrosis, scleroderma, endometriosis, polycystic ovary syndrome and cirrhosis of the liver, the method comprising the administration of the compound of formula (I) as defined in claim 1 or the anion of said compound, or a composition comprising said compound and an additional compound or agent, to a patient in need of such treatment and/or prevention.

24. A method of treating and/or preventing infections, the method comprising the administration of the compound of formula (I) as defined in claim 1 the anion of said compound, or a composition comprising said compound and an additional compound or agent, to a patient in need of such treatment and/or prevention.

25. A method of treating and/or preventing metastases, the method comprising the administration of the compound of formula (I) as defined in claim 1 the anion of said compound, or a composition comprising said compound and an additional compound or agent, to a patient in need of such treatment and/or prevention.

26. A method of treating and/or preventing a cell proliferation disorder, the method comprising the administration of the compound of formula (I) as defined in claim 1 or the anion of said compound, or a composition comprising said compound and an additional compound or agent, to a patient in need of such treatment and/or prevention.

27. A radiotherapy method that comprises a step of using the compound of formula (I) as defined in claim 1 or the anion of said compound, or a composition comprising said compound and an additional compound or agent, as a radiotherapy sensitizing agent.

28. A pharmaceutical composition comprising the compound of formula (I) as defined in claim 1 or the anion of said compound, or a composition comprising said compound and an additional compound or agent.

29. A kit-of-parts comprising: the compound of formula (I) as defined in claim 1 or the anion of said compound, or a composition comprising said compound and an additional compound or agent, optionally in admixture with a pharmaceutically acceptable adjuvant, diluent or carrier.

30. An apoptotic agent comprising the compound of formula (I) as defined in claim 1 or the anion of said compound, or a composition comprising said compound and an additional compound or agent.

31. A method of inducing thiol oxidation comprising administering the compound of formula (I) as defined in claim 1 or the anion of said compound, or a composition comprising said compound and an additional compound or agent, optionally in combination with reactive oxygen species (ROS).
